(19) United States
(12) Patent Application Publication
(10) Pub. No.: US 2025/0046423 A1
(43) Pub. Date: Feb. 6, 2025

(54) SYSTEMS AND METHODS FOR PROVIDING INDIVIDUALIZED NUTRITIONAL RECOMMENDATIONS FOR INTERMITTENT FASTING

(71) Applicant: SOCIETE DES PRODUITS NESTLE S.A., Vevey (CH)

(72) Inventors: PALOMA ELORTEGUI PASCUAL, Epalinges (CH); ERIC ANTOINE SCUCCIMARRA, Lausanne (CH); LOLITA BAZAROVA, Lausanne 26 (CH)

(21) Appl. No.: 18/705,354

(22) PCT Filed: Oct. 26, 2022
(86) PCT No.: PCT/EP2022/079996

§ 371 Date: Apr. 26, 2024

Related U.S. Application Data
(60) Provisional application No. 63/272,253, filed on Oct. 27, 2021.

(51) Int. Cl. G16H 20/60; G16H 10/60

(52) U.S. Cl. CPC G16H 20/60; G16H 10/60

(57) ABSTRACT

Systems and methods provide individualized nutritional recommendations before, during and/or after an intermittent fasting (IF) diet. In several embodiments, the individualized nutritional recommendations are diets, menus and recipes for before, during and/or alter an intermittent fasting (IF) diet. In several embodiments, the nutritional recommendations are delivered by a computer-implemented system. In several embodiments, the individual is administered and/or consumes nutrients according to the nutritional recommendations, for example calcium, preferably one or more calcium salts, such as calcium acetate, calcium carbonate, calcium chloride, calcium citrate, calcium gluconate, calcium lactate or mixtures thereof; and/or a fiber supplement, preferably those containing one or more of wheat dextrin, methylcellulose, psyllium husk, polycarbophil, guar fiber, glucomannan or β-glucans.

### TECHNICAL FIELD

The present disclosure generally relates to systems and methods for providing individualized nutritional recommendations before, during and/or after an intermittent fasting (IF) diet. In a preferred embodiment, the nutritional recommendations are diets, menus and recipes for before, during and/or after an IF diet, preferably a time-restricted feeding (TRF) regimen or an alternate day fasting (ADF) regimen. In a preferred embodiment, the nutritional recommendations are delivered by a computer-implemented system.

### BACKGROUND

Intermittent fasting (IF) is a generic term to describe dietary patterns that include a period where little or no energy intake is consumed, alternating with a feeding-sometimes referred to as feasting-period.

One of the most popular styles of IF is the time-restricted feeding (TRF) regimen in which consumption of food is only allowed during a specified period of time per day, e.g., people on a 16:8 regimen fast for 16 hours and eat for 8 hours each day. Another popular style of IF is alternate day fasting (ADF) in which 24-hour fasting periods alternate with 24-hours of ad libitum intake. There are three common versions of ADF: (1) strict alternate day fasting in which people alternate one day of eating with one day of fasting; no calories are consumed on the fasting day, but non-caloric beverages (e.g., water, infusions, tea, coffee) may be consumed; (2) modified alternate day fasting in which people alternate one day of eating with one day of a modified fast, consuming foods and beverages with a limited number of calories (e.g., ≤500 kcal) on the fasting day; and (3) a 5:2 regimen in which people fast two out of every seven days, including modifications in which a limited number of calories can be consumed two out of seven days.

Generally used as a weight loss regimen, IF has also been purported to have other health benefits including glycemic control and cardiovascular health, based on animal and human research. Efficacy of IF on health parameters varies widely in the literature, most likely due to the variability in study subjects and study designs.

A person following an IF diet typically will not be aware of possible nutrient inadequacies stemming from the diet or the amounts of those inadequacies. These two points cannot be easily answered by existing clinical trials because the real-life eating patterns may differ from the dietary choices given in a clinical setting, under controlled conditions and constant supervision, therefore, the nutrients at risk in clinical settings can differ from real-life settings. In addition, most randomized controlled trials were performed in obese or overweight populations; and the study sizes and number of subjects in previous studies are generally small. Finally, nutritional needs are dependent on age, gender, height, weight and individual conditions (pregnancy, lactation, etc.).

Furthermore, constant daily reporting and analysis of individual's diet is not practicable because daily recording is tedious, people forget to report individual items and eating occasions, they do not have access to nutritional databases, and they lack the expertise needed to identify nutrient inadequacies.

### SUMMARY

Several digital mobile phone applications provide recipe ideas for an intermittent fasting regimen. However, these applications do not account for risks of nutritional inadequacies, and they are not tailored to age, gender, physical activity, health conditions, etc.

Moreover, individual food item recommendations before, during and/or after an intermittent fasting (IF) diet are often difficult for individual users to implement, because individual food item recommendations are not considered in the context of an entire diet, menus, or recipes over different meals throughout a day or over several weeks. The methods and systems disclosed herein advantageously implement food recommendations into user-friendly, practical, and actionable diets, menu plans or recipes that can be adapted to specific individual needs and preferences before, during and/or after an IF diet. In this way, the individual user is provided clear guidance on how to implement nutritional recommendations in their daily diet, menus and recipes.

Indeed, the present disclosure provides new nutritional recommendations and innovative methods for personalized nutrient, dietary and lifestyle recommendations for individuals who are following or planning to follow an IF regimen, such as a TRF regimen (e.g., 16:8, 20:4, or 12:12) or an ADF regimen (e.g., strict ADF, modified ADF, or 5:2). In some embodiments, the nutritional recommendations are generated and/or used less one month before performing the IF regimen, for example less than one week before performing the IF regimen. Additionally or alternatively, the nutritional recommendations are generated and/or used at least the majority of the days during the performing of the IF regimen, for example daily during the performing of the IF regimen. Additionally or alternatively, the nutritional recommendations are generated and/or used for at least one week after performing the IF regimen, for example for at least one month after performing the IF regimen.

One or more embodiments disclosed herein advantageously determine nutritional recommendations to maintain the recommended daily allowance for an overall healthy diet before, during and/or after an IF diet. In particular, the requirements for micronutrients are observed, for example, the micronutrient requirements for daily vitamins and minerals.

A further advantage of one or more embodiments disclosed herein is to implement individual user dietary preferences, such as, gluten-free, lactose-free, Mediterranean, paleo, vegan or vegetarian diets, when constructing the menu plans in the nutritional recommendations before, during and/or after an IF diet.

In an embodiment, the nutritional solutions include foods, beverages and/or dietary supplements thus recommended in the context of defined IF dietary pattern, lifestyle and dietary rules.

In some embodiments, the present disclosure provides a method of minimizing or preventing nutrient inadequacy in an individual performing an IF regimen, wherein the method comprises using any of the methods and systems disclosed herein, and preferably the method comprises administering one or more of nutrients, food containing nutrients, and/or amounts thereof according to the recommendations during a time period that is before, during and/or after the IF regimen. Non-limiting examples of suitable nutrients for minimizing or preventing nutrient inadequacy in an individual performing an IF regimen include calcium, preferably one or more calcium salts, such as calcium acetate, calcium carbonate, calcium chloride, calcium citrate, calcium gluconate, calcium lactate or mixtures thereof; and/or a fiber supplement, preferably those containing one or more of wheat dextrin, methylcellulose, psyllium husk, polycarbophil, guar fiber, glucomannan or β-glucans.

Additional features and advantages are described herein and will be apparent from the following Figures and Detailed Description.

### BRIEF DESCRIPTION OF DRAWINGS

FIG. 1 is a block diagram of an example computer-implemented system for nutritional recommendations before, during and/or after an IF diet, according to an embodiment provided by the present disclosure.

FIG. 2 is a screenshot of individual biometric data for a typical user before, during and/or after an IF diet, according to an embodiment provided by the present disclosure.

FIGS. 3A-3C show a sample menu plan for one day before, during and/or after an IF diet, according to an embodiment provided by the present disclosure. FIG. 3A is a screenshot showing the nutritional content of a menu plan for one day. FIG. 3B is a screenshot showing breakfast and lunch suggestions. FIG. 3C is a screenshot showing shows dinner and snack suggestions. Meal or food item selections can be marked with a tag to show which selections are nutritionally healthy. In the snack selection, there is the possibility to select alternative items to substitute. In this example, the pistachios can be selected or alternatively pistachios roasted without salt may be selected. The images were marked with a nutritional symbol 1202 for those tagged as nutritionally healthy, because they contained one of the nutritionally healthy rules. The arrow symbol 1204 allows the user to interchange the recipes or dishes created automatically by the engine. The meal nutritional score, marked with a symbol, “My Menu IQ” 1206, is displayed to assign the meal nutritional score out of 100 for each meal occasion.

FIGS. 4A and 4B show a sample menu plan for one day before, during and/or after an IF diet, according to an embodiment provided by the present disclosure. FIG. 4A is a screenshot showing the ingredients and the amounts in the recipe. FIG. 4B is a screenshot showing the instructions how to make the recipe.

FIG. 5 is a schematic diagram of an example recommendation system for nutritional recommendations before, during and/or after an IF diet, according to an embodiment provided by the present disclosure.

FIG. 6 is a workflow diagram for building a nutritionally healthy menu planner before, during and/or after an IF diet, according to an embodiment provided by the present disclosure.

FIG. 7 is a graph of calcium intake ranges comparing simulated baseline intakes with 16:8 TRF for males. The boxes show the 25th percentile (lower bound) and 75th percentile (upper bound) of intakes for each diet. The bar in the middle of each box is the median intake. Median intakes at baseline exceed the RDA for calcium, but above the EAR. The 16:8 TRF regimen has median intakes lower than the median at baseline.

FIG. 8 is a graph of calcium intake ranges comparing simulated baseline intake with 16:8 TRF for females. The boxes show the 25th percentile (lower bound) and 75th percentile (upper bound) of intakes for each diet. The bar in the middle of each box is the median intake. Median intakes at baseline exceed the RDA for calcium, but above the EAR. The 16:8 TRF regimen has median intakes lower than the median at baseline.

FIG. 9 is a graph of fiber intake ranges comparing simulated baseline intakes with 16:8 TRF for males. The boxes show the 25th percentile (lower bound) and 75th percentile (upper bound) of intakes for each diet. The bar in the middle of each box is the median intake. Fiber intakes at baseline and for 16:8 TRF diet are below the AI.

FIG. 10 is a graph of fiber intake ranges comparing simulated baseline intakes with 16:8 TRF for females. The boxes show the 25th percentile (lower bound) and 75th percentile (upper bound) of intakes for each diet. The bar in the middle of each box is the median intake. The median intakes of fiber for females at baseline is above the AI. Median intakes for fiber 16:8 TRF are below the AI.

FIG. 11 is a graph of calcium intake ranges comparing simulated baseline intakes with strict ADF, modified ADF, and 5:2 for males. The boxes show the 25th percentile (lower bound) and 75th percentile (upper bound) of intakes for each diet. The bar in the middle of each box is the median intake. All ADF regimens have median intakes lower than the median at baseline. Median intakes at baseline exceed the RDA for calcium. Median intakes of calcium for 5:2 also exceeds the RDA, but median intakes for both strict ADF and modified ADF and below the RDA.

FIG. 12 is a graph of calcium intake ranges comparing simulated baseline intake with strict ADF, modified ADF, and 5:2 for females. The boxes show the 25th percentile (lower bound) and 75th percentile (upper bound) of intakes for each diet. The bar in the middle of each box is the median intake. All ADF regimens have median intakes lower than the median at baseline. Median intakes at baseline are below the RDA, but above the EAR. Median intakes of calcium for all ADF diets are below the EAR.

FIG. 13 is a graph of fiber intake ranges comparing simulated baseline intakes with strict ADF, modified ADF, and 5:2 for males. The boxes show the 25th percentile (lower bound) and 75th percentile (upper bound) of intakes for each diet. The bar in the middle of each box is the median intake. Fiber intakes at baseline and for all ADF diets are below the AI. Median fiber intakes are lowest for strict ADF, followed by modified ADF.

FIG. 14 is a graph of fiber intake ranges comparing simulated baseline intakes with strict ADF, modified ADF, and 5:2 for females. The boxes show the 25th percentile (lower bound) and 75th percentile (upper bound) of intakes for each diet. The bar in the middle of each box is the median intake. The median intakes of fiber for females at baseline is above the AI. Median intakes for fiber on all ADF diets are below the AI, with lowest intakes in strict ADF.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

Examples of types of Time Restricted Feeding (TRF) include the following:
(1) 12:12—fasting for 12 hours and ad libitum eating for 12 hours,
(2) 16:8—fasting for 16 hours and ad libitum eating for 8 hours, and
(3) 20:4—fasting for 20 hours and ad libitum eating for 4 hours.

Types of Alternate Day Fasting (ADF) include the following:
(1) Strict Alternate Day Fasting where people alternate one day of eating with one day of fasting. In this regimen, no calories are consumed on the fasting day, but non-caloric beverages (e.g., water, infusions, tea, coffee) may be consumed.
(2) Modified Alternate Day Fasting where people alternate one day of eating with one day of a modified fast, consuming foods and beverages with a limited number of calories (e.g. ≤500 kcal) on the fasting day.
(3) 5:2 regimen where people fast two out of every seven days. The 5:2 regimen also includes modifications where on two out of seven days, a limited number of calories can be consumed.

Within the context of the present disclosure, “nutrients” are substances needed for health, growth, development and functioning of an organism, including: macronutrients (e.g., protein, carbohydrates, fats) and their components (e.g., amino acids, sugars, starches, fatty acids, etc.); micronutrients (e.g., vitamins, minerals); other food components (fiber, cholesterol, bioactive phytochemicals, alcohol, etc.); and water contained in foods and beverages.

The term “composition” can mean a food, beverage, dietary supplement, complete nutrition or oral nutritional supplement (ONS) or medical food composition, or mixture thereof.

Within the context of the present disclosure, the terms “food,” “food product” and “food composition” mean a product or composition that is intended for ingestion by an individual such as a human and provides nutritional support to an organism, including those that provide energy, nutrients, and water. The compositions of the present disclosure, including the many embodiments described herein, can comprise, consist of, or consist essentially of one or more of the nutrients listed above, as well as any additional or optional ingredients or components safe for human consumption and otherwise useful in a diet.

Within the context of the present disclosure, the term “beverage,” “beverage product” and “beverage composition” mean a potable liquid product or composition for ingestion by an individual such as a human and provides water and may also include one or more nutrients and other ingredients safe for human consumption to the individual. The compositions of the present disclosure, including the many embodiments described herein, can comprise, consist of, or consist essentially of one or more of the nutrients listed above, as well as any additional or optional ingredients, components safe for human consumption and otherwise useful in a diet.

Within the context of the present disclosure, “dietary supplements” are products taken by mouth that contain one or more dietary ingredient, such as vitamins, minerals, amino acids, fatty acids, fibers and/or herbs and other botanical ingredients used to supplement the diet. Dietary supplements come in many forms and may be available as tablets, capsules, powders, liquids, and formulated into specific foods, such as “energy” bars.

As used herein, “complete nutrition” contains sufficient types and levels of macronutrients (protein, fats and carbohydrates), micronutrients, and other food components to be sufficient to be a sole source of nutrition for the subject to which the composition is administered. Individuals can receive 100% of their nutritional requirements from such complete nutritional compositions.

Within the context of the present disclosure, the term “Dietary Reference Intakes (DRIs)” indicates a set of reference values used to plan and assess the nutrient intakes of healthy people. The DRIs were established by the United States and Canadian governments, and published by the National Academies of Sciences, Engineering, and Medicine (NASEM; formerly called Institute of Medicine (IOM); https://www.nal.usda.gov/fnic/dri-nutrient-reports). Within the context of the present disclosure, the terms used to describe the DRIs (Institute of Medicine (US) Food and Nutrition Board. Dietary Reference Intakes: A Risk Assessment Model for Establishing Upper Intake Levels for Nutrients. Washington DC, USA: National Academies Press; 1998. What are Dietary Reference Intakes?) are as follows:

Recommended Dietary Allowance (RDA) indicates the average daily intake level of a nutrient considered sufficient to meet the requirements of 97.5% of healthy individuals. RDAs vary by gender, age, and whether a woman is pregnant or lactating. RDAs are calculated based on the Estimated Average Requirements (EAR) and is usually approximately 20% higher than the EAR.

Adequate Intake (AI) for a nutrient is the amount estimated to meet or exceed the amount needed to maintain adequate nutrition for most people in a specific age or gender group. An AI is set instead of an RDA when there is insufficient evidence to calculate an EAR.

Tolerable Upper Intake Level (UL) is the highest amount of daily nutrient intake considered to pose no risk of adverse health effects for most people. Consuming more than the UL increases the risk of adverse effects from over-consumption.

Estimated Average Requirement (EAR) for nutrients are calculated to meet the requirements of 50% of the people in a specific age and gender group. The EAR is required to establish an RDA. If the standard deviation (SD) of the EAR is available and the requirement for the nutrient is symmetrically distributed, the RDA is set at two SDs above the EAR:

RDA=EAR+2SD (EAR)

If data about variability in requirements are insufficient to calculate a SD, a coefficient of variation (CV) for the EAR of 10 percent is assumed, unless available data indicate a greater variation in requirements. If 10 percent is assumed to be the CV, then twice that amount when added to the EAR is defined as equal to the RDA. The resulting equation for the RDA is: RDA=1.2 (EAR).

Different national and regional authorities have different dietary reference values. For example, The European Food Safety Authority (EFSA) refers to the collective set of information as Dietary Reference Values (DRV), with Population Reference Intake (PRI) instead of RDA, and Average Requirement instead of EAR. AI and UL are defined the same as in United States, but values may differ (EFSA Panel on Dietetic Products, Nutrition, and Allergies (NDA). Scientific Opinion on Principles for Deriving and Applying Dietary Reference Values. EFSA J. 2020; 8(3):1458. https://doi.org/10.2903/j.efsa.2010.1458). These standards and values are also used in the context of this disclosure.

Within the context of the present disclosure, the terms “nutrient inadequacy” or “dietary inadequacy” indicates that the total daily dietary intake of a nutrient in a certain individual is below the Estimated Average Requirements (EARs) for said individual and/or below well-established nutritional requirements.

Within the context of the present disclosure, the expression “prevent nutrient inadequacy” should be understood to include prevention of inadequacies of the nutrient or nutrients, as well as reduction of the risk of nutrient inadequacies in the individual following an ADF diet.

As used in this disclosure and the appended claims, the singular forms “a,” “an” and “the” include plural referents unless the context clearly dictates otherwise. Thus, for example, reference to “a nutrient” or “the nutrient” encompass both an embodiment having a single nutrient and an embodiment having two or more nutrients.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of inositol or sorbitol” and “inositol and/or sorbitol” should be interpreted as “inositol without sorbitol,” or “sorbitol without inositol,” or “inositol without sorbitol.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual.

A “subject” or “individual” is a mammal, preferably a human.

### Embodiments

One or more embodiments of the disclosed system satisfy the general goal, given a particular IF diet (preferably a TRF regimen or an ADF regimen), to recommend a set of foods or menus or recipes in order to maintain or improve the overall nutritional health of the individual. The nutritional health depends on the general characteristics of the individual (gender, age, weight, body measurements, physical activity level, and/or other health-related conditions like pregnancy or lactation etc.), and the recommendations to maintain or improve the nutritional health likewise depend on characteristics of the individual.

In one or more embodiments, the system disclosed herein calculates and displays recommendations of food items, menus or recipes for before, during and/or after an IF diet (preferably a TRF regimen or an ADF regimen). In these embodiments, the system determines and stores one or more indications of the needs of the individual for whom the recommendations are being calculated, for an individual over a given period of time such as a meal, an entire day, a week or a month.

The system then enables the user to indicate consumables (such as food items) that he or she has consumed or plans to consume. For each indicated food item, a database or data store of the disclosed system stores an indication of the nutrient content, particularly the micronutrient content, per amount of that food item or menu or recipe. The system uses the nutritional content information, multiplied by the amount of food item consumed over time, to determine the total nutritional intake over a time period for that particular food item or menu or recipe.

In one or more embodiments, the disclosed system provides a recommendation function, wherein the system suggests combinations of foods or menus or recipes that will result in an improved or optimal nutritional menu. For example, if a user accesses the system after breakfast and indicates the foods he or she had for breakfast, the disclosed system may calculate a score for the breakfast foods, but may also determine what nutrients would need to be consumed over the remainder of the day, as well as how much energy to consume over the remainder of the day, for the individual to consume nutrients and energy in the optimal ranges for that day to have an overall nutritional health before, during and/or after an IF diet (preferably a TRF regimen or an ADF regimen). In this embodiment, the system uses these calculated nutrient amounts to determine combinations of food that can be consumed throughout the remainder of the day to ensure that the individual's nutritional goals are achieved as fully as possible while still consuming a number of calories within that individual's optimal caloric intake range. Thus, the system disclosed herein can operate not only as a tracking system, but also as a recommendation engine to recommend consumables to help individuals reach their nutritional goal during an IF diet (preferably a TRF regimen or an ADF regimen).

The term “nutrient” is used repeatedly herein. In some embodiments, the term “nutrient” as used herein refers to compounds having a beneficial effect on the body, e.g., to provide energy, growth or health. The term includes organic and inorganic compounds. As used herein the term nutrient may include, for example, macronutrients, micronutrients, essential nutrients, conditionally essential nutrients and phytonutrients. These terms are not necessarily mutually exclusive. For example, certain nutrients may be defined as either a macronutrient or a micronutrient depending on the particular classification system or list.

In various embodiments, the term “macronutrient” is used herein consistent with its well-understood usage in the art, which generally encompasses nutrients required in large amounts for the normal growth and development of an organism. Macronutrients in these embodiments may include, but are not limited to, carbohydrates, fats, proteins, amino acids and water. Certain minerals may also be classified as macronutrients, such as calcium, chloride, or sodium.

In various embodiments, the term “micronutrient” is used herein consistent with its well understood usage in the art, which generally encompasses compounds having a beneficial effect on the body, e.g., to help provide energy, growth or health, but which are required in only minor or trace amounts. The term in such embodiments may include organic compounds and/or inorganic compounds, e.g., individual amino acids, nucleotides and fatty acids; vitamins, antioxidants, minerals, trace elements, e.g. iodine, and electrolytes, e.g. sodium, and salts thereof, including sodium chloride.

In several embodiments, micronutrients are calculated, in particular vitamins and minerals, for a particular food, menu, or recipe of nutritionally healthy items during an IF diet (preferably a TRF regimen or an ADF regimen). The system may tag such items as “healthy” with an icon so that these items are readily identifiable to the user.

In one or more embodiments, menus and recipes are selected based on these food or nutrient groups per time period in order to obtain a nutritionally healthy menu or recipe before, during and/or after an IF diet (preferably a TRF regimen or an ADF regimen). The system allows for substitution of food items, menus and recipes in order to build meals throughout the day.

In one or more embodiments, the menus and recipes are based on the amount of the food or nutrient groups needed in order to have a balanced diet overall as well as being nutritionally healthy before, during and/or after an IF diet (preferably a TRF regimen or an ADF regimen).

In one or more embodiments, the system considers individual user preferences, such as gluten-free, lactose-free, Mediterranean diet, paleo diet, vegan diet, vegetarian diet and other specific diets.

Individual user likes or dislikes can be stored within the system so that some recommendations of food items, menus and recipes are avoided. The system may also store the frequency of such food items, menus and recipes so they can be varied in order to avoid boredom from the user in having the same menu or recipe each day.

In one or more embodiments, one or more devices carried by the user could provide real-time information to the system when the user is in a food purchasing establishment such as a grocery store or a restaurant. Devices such as RFID readers, NFC readers, wearable camera devices, and mobile phones could receive or determine foods that are available to a user at a particular grocery store or restaurant (such as by scanning RFID tags, reading bar codes, or determining the physical location of a user). The disclosed system could then make nutritionally healthy recommendations based on the foods that could be immediately purchased or consumed by the user.

In some embodiments, when a user sits down at a restaurant, the disclosed system may push information to the user's mobile phone recommending that the user select certain items from the menu to optimize the user's individual nutritionally healthy menu for a given time period before, during and/or after an IF diet (preferably a TRF regimen or an ADF regimen). In some embodiments, a voice recognition feature recognizes inputs provided vocally by a user. In such embodiments, the voice recognition system listens as a user orders at a restaurant; additionally or alternatively, the voice recognition system enables the user to speak directly the items the user has consumed or will consume. In some embodiments, the disclosed system uses geolocation to provide appropriate exercise recommendations based on the user's location. For example, an application executed on a user's phone, tablet, or computer could provide the user different activity tips if the user is at work, in a gym, or at home (e.g., different activity tips in a chat box).

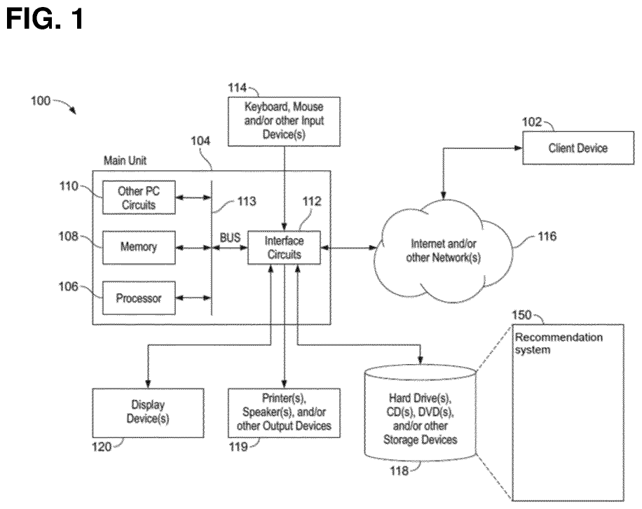

Referring now to FIG. 1, a block diagram is illustrated showing an example of the electrical systems of a host device 100 usable to implement at least portions of the computerized recommendation system disclosed herein.

In one or more embodiments, the host device 100 comprises one or more servers and/or other computing devices that provide some or all of the following functions: (a) enabling access to the disclosed system by remote users of the system; (b) serving web page(s) that enable remote users to interface with the disclosed system; (c) storing and/or calculating underlying data, such as recommended caloric intake ranges, recommended nutrient consumption ranges, and nutrient content of foods, needed to implement the disclosed system; (d) calculating and displaying component; and/or (e) making recommendations of foods, menus or recipes or other consumables that can be consumed to help individuals reach an optimal nutritional health.

In the example architecture illustrated in FIG. 1, the host device 100 includes a main unit 104 which preferably includes one or more processors 106 electrically coupled by an address/data bus 113 to one or more memory devices 108, other computer circuitry 110, and/or one or more interface circuits 112. The one or more processors 106 may be any suitable processor, such as a microprocessor from the INTEL PENTIUM® or INTEL CELERON® family of microprocessors. PENTIUM® and CELERON® are trademarks registered to Intel Corporation and refer to commercially available microprocessors. Additionally or alternatively, other commercially-available or specially-designed microprocessors may be used as processor 106. In one or more embodiments, the processor 106 is a system on a chip (“SOC”) designed specifically for use in the disclosed system.

In one or more embodiments, the host device 100 includes memory 108. The memory 108 preferably includes volatile memory and non-volatile memory. Preferably, the memory 108 stores one or more software programs that interact with the hardware of the device 100 and with one or more other devices in the system as described below. In addition or alternatively, the programs stored in the memory 108 may interact with one or more client devices such as client device 102 (discussed in detail below) to provide the one or more client devices with access to media content stored on the host device 100. The programs stored in the memory 108 may be executed by the processor 106 in any suitable manner.

The interface circuit(s) 112 may be implemented using any suitable interface standard, such as an Ethernet interface and/or a Universal Serial Bus (USB) interface. One or more input devices 114 may be connected to the interface circuit 112 for entering data and commands into the main unit 104. For example, the input device 114 may be a keyboard, mouse, touch screen, track pad, track ball, isopoint, and/or a voice recognition system. In one embodiment, wherein the host device 100 is designed to be operated or interacted with only via remote devices, the host device 100 may not include input devices 114. In other embodiments, input devices 114 include one or more storage devices, such as one or more flash drives, hard disk drives, solid state drives, cloud storage, or other storage devices or solutions, which provide data input to the host device 100.

One or more storage devices 118 may also be connected to the main unit 104 via the interface circuit 112. For example, a hard drive, CD drive, DVD drive, flash drive, and/or other storage devices may be connected to the main unit 104. The storage devices 118 may store any type of data used by the device 100, including data regarding preferred nutrient ranges, data regarding nutrient contents of various food items, data regarding users of the system, data regarding previously-generated dietary intake scores, data regarding previously-generated menus, recipes or meals, individual user preferences for menus, recipes or meals, frequency of preferences for menus, recipes or meals, data regarding ideal energy intake, data regarding past energy consumption, and any other appropriate data needed to implement the recommendation system 150.

In some embodiments, the recommendation system 150 may store different database modules which include: a food database module; a menu database module (for example with: breakfast, lunch, dinner and snacks); a recipe database module; a dietary constraints module (for example with gluten-free, lactose-free, Mediterranean diet, paleo diet, vegetarian diet, vegan diet recommendations based on the dietary constraints); a nutrient scoring module (for example for determining the macronutrient or micronutrient score per menu, per recipe or per day); and/or an optimization module.

Alternatively or in addition, the storage devices 118 may be implemented as cloud-based storage, such that access to the storage devices 118 occurs via an internet or other network connectivity circuit, such as an Ethernet circuit 112.

One or more displays 120, and/or printers, speakers, or other output devices 119 may also be connected to the main unit 104 via the interface circuit 112. The display 120 may be a liquid crystal display (LCD), a suitable projector, or any other suitable type of display. The display 120 generates visual representations of various data and functions of the device 100 during operation of the device 100. For example, the display 120 may be used to display information about the database of preferred nutrient ranges, a database of nutrient contents of various food items, a database of users of the system, a database of previously-generated menus, recipes or meals, and/or databases to enable an administrator at the device 100 to interact with the other databases described above.

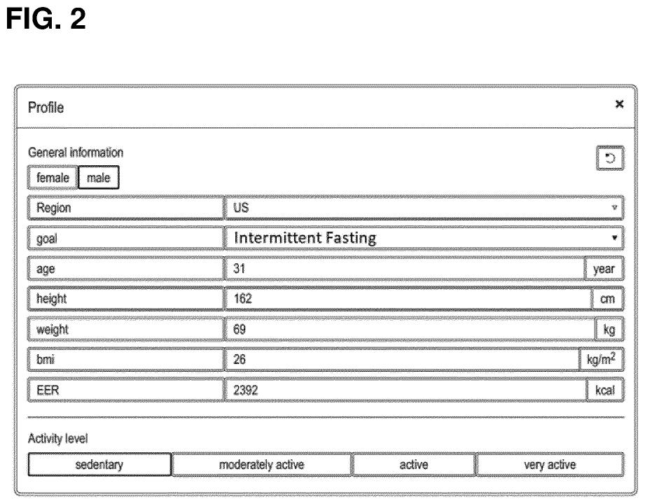

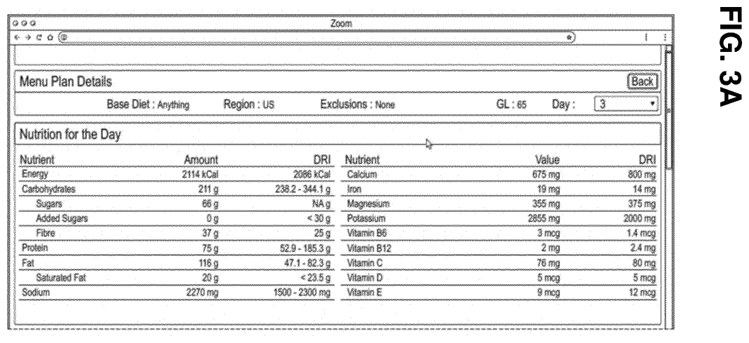

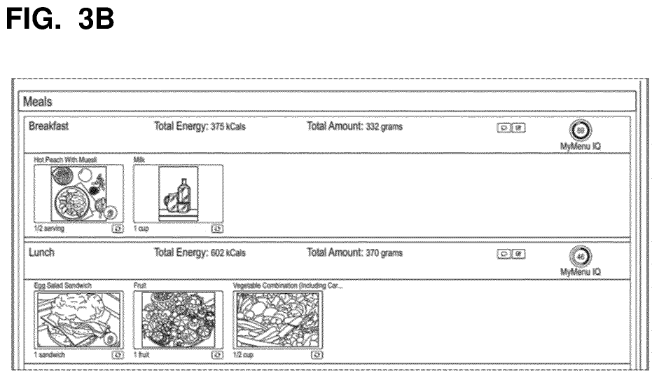

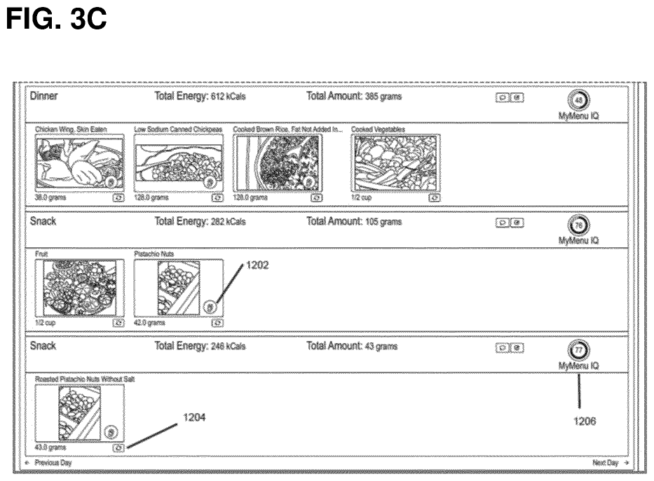

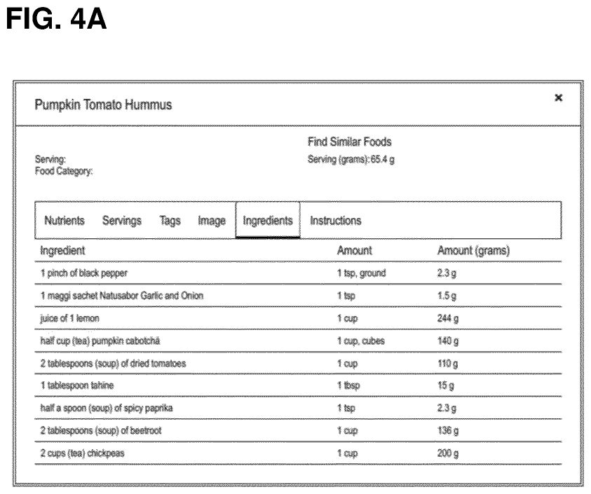

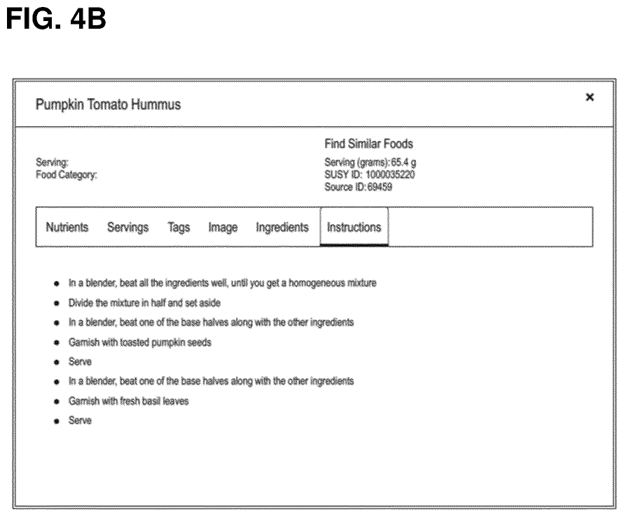

For example, FIG. 2 shows individual user information. FIGS. 3A-3C show a typical menu plan for one day. FIGS. 4A and 4B show a typical nutritionally healthy recipe and instructions how to make it.

In the illustrated embodiment, the users of the recommendation system 150 interact with the host device 100 using a suitable client device, such as the client device 102. The client device 102 is any device that can access content provided or served by the host device 100. For example, the client device 102 may be any device that can run a suitable web browser to access a web-based interface to the host device 100. Alternatively or in addition, one or more applications or portions of applications that provide some of the functionality described herein may operate on the client device 102, in which case the client device 102 is required to interface with the host device 100 merely to access data stored in the host device 100, such as data regarding healthy nutrient ranges or nutrient content of various food items.

In one or more embodiments, this connection of devices (i.e., the host device 100 and the client device 102) is facilitated by a network connection 116, for example over the Internet and/or other networks, as illustrated in FIG. 1. The network connection 116 may comprise any suitable network connection, such as an Ethernet connection, a digital subscriber line (DSL), a Wi-Fi connection, a cellular data network connection, a telephone line-based connection, a connection over coaxial cable, and/or another suitable network connection.

In some embodiments, the host device 100 is a device that provides cloud-based services, such as cloud-based authentication and access control, storage, streaming, and feedback provision. In this embodiment, the specific hardware details of the host device 100 are not important to the implementer of the disclosed system; instead, in such an embodiment, the implementer of the disclosed system utilizes one or more Application Programmer Interfaces (APIs) to interact with the host device 100 in a convenient way, such as to enter information about the user's demographics to help determine healthy nutritional ranges, to enter information about consumed foods, and other interactions described in more detail below.

Access to the host device 100 and/or the client device 102 may be controlled by appropriate security software or security measures. An individual user's access can be defined by the host device 100 and limited to certain data and/or actions, such as selecting various menus or recipes or viewing calculated scores, according to the individual's identity. Other users of the host device 100 or the client device 102 may be allowed to alter other data, such as weighting, sensitivity, or healthy range values, depending on those users' identities. Accordingly, users of the system may be required to register with the host device 100 before accessing the content provided by the disclosed system.

In a preferred embodiment, each of the one or more client devices 102 has a similar structural or architectural makeup to that described above with respect to the host device 100. That is, each of the one or more client devices 102 can include a display device, at least one input device, at least one memory device, at least one storage device, at least one processor, and at least one network interface device. The one or more client devices 102 can using such components, which are common to well-known desktop, laptop, or mobile computer systems (including smart phones, tablet computers, and the like) to facilitate interaction among and between each of the one or more client devices 102 by users of the respective systems.

In some embodiments, the host device 100 and/or the one or more client devices 102 may be implemented as a plurality of different devices. For example, the host device 100 may be implemented as a plurality of server devices operating together to implement the media content access system described herein. In some embodiments, one or more additional devices not shown in FIG. 1 interact with the host device 100 to enable or facilitate access to the system disclosed herein. For example, the host device 100 can communicate via the network connection 116 with one or more public, private, or proprietary repositories of information, such as public, private, or proprietary repositories of nutritional information, nutrient content information, menu planners, recipe databases, healthy range information, energy information, environmental impact information, or the like.

In some embodiments, the disclosed system does not include a client device 102. In this embodiment, the functionality described herein is provided on the host device 100, and the user of the system interacts directly with the host device 100 using the input devices 114, the display device 120, and the output devices 119. In this embodiment, the host device 100 preferably provides some or all of the functionality described herein as user-facing functionality.

In various embodiments, the system disclosed herein is arranged as a plurality of modules, wherein each module performs a particular function or set of functions. The modules in these embodiments can be one or more software modules executed by a general purpose processor; one or more software modules executed by a special purpose processor; on or more firmware modules executing on an appropriate, special-purpose hardware device; and/or one or more hardware modules, such as application specific integrated circuits (“ASICs”), that perform the functions recited herein entirely with circuitry. In embodiments where specialized hardware is used to perform some or all of the functionality described herein, the disclosed system may use one or more registers or other data input pins to control settings or adjust the functionality of such specialized hardware.

A user goal to consume a nutritionally healthy diet may be examined over time to detect potential problematic menus or meals within the diet. The system can be used to then identify the recommended shifts needed in the food items, menus or recipes in order to get closer to the recommended amounts. In some embodiments, the system and methods disclosed herein can be used by one or more of a nutritionist, a health-care professional, or an individual users (e.g., a user of wearable devices such as smart watches or fitness trackers).

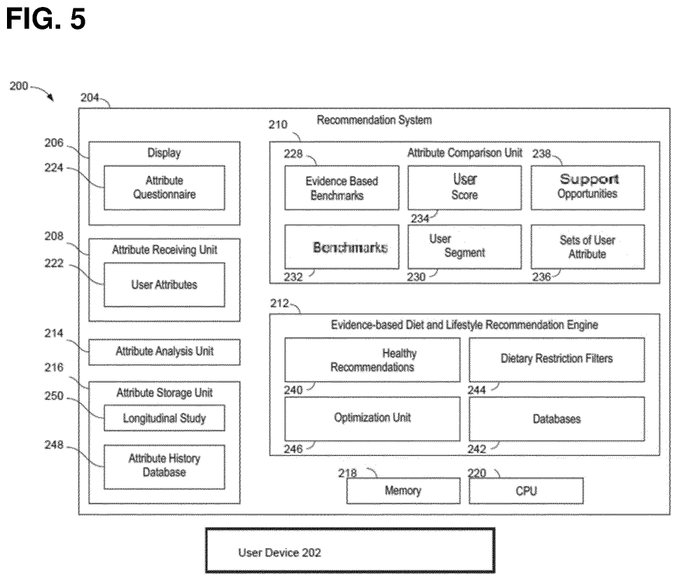

FIG. 5 illustrates a nutritional recommendation system 200 according to an embodiment provided by the present disclosure. The nutritional recommendation system 200 preferably includes a user device 202 and/or a recommendation system 204. In some embodiments, the recommendation system 204 can function as the recommendation system 150. The user device 202 may be implemented as a computing device, such as a computer, smartphone, tablet, smartwatch, or other wearable through which an associated user can communicate with the recommendation system 204. The user device 202 may also be implemented as, e.g., a voice assistant configured to receive voice requests from a user and to process the requests either locally on a computer device proximate to the user or on a remote computing device (e.g., at a remote computing server).

The recommendation system 204 preferably includes one or more of a display 206, an attribute receiving unit 208, an attribute comparison unit 210, an evidence-based diet and lifestyle recommendation engine 212, an attribute analysis unit 214, an attribute storing unit 216, a memory 218, or a processing unit 220. In some embodiments, a display 206 may additionally or alternatively be located within the user device 202. In an example embodiment, the recommendation system 204 may be configured to receive a request for a plurality of nutritionally healthy recommendations 240. For example, a user may install an application on the user device 202 that requires the user to sign up for a recommendation service. By signing up for the service, the user device 202 may send a request for the nutritionally healthy recommendations 240. Additionally or alternatively, the user may use the user device 202 to access a web portal using user-specific credentials. Through this web portal, the user may cause the user device 202 to request nutritionally healthy recommendations from the recommendation system 204.

In some embodiments, the recommendation system 204 may be configured to request and receive a plurality of user attributes 222. For example, the display 206 may be configured to present an attribute questionnaire 224 to the user. The attribute receiving unit 208 may be configured to receive the user attributes 222. In some embodiments, the attribute receiving unit 208 may receive a plurality of answers 226 based on the attribute questionnaire 224, and based on the plurality of answers, determine the plurality of user attributes 222. For example, the attribute receiving unit 208 may receive answers to the attribute questionnaire 224 suggesting that the diet of the user is equivalent to the recommended dietary allowance (“RDA”) and then determine the user attributes 222 to be equivalent to the RDA, of Vitamin K per day. Additionally or alternatively, the attribute receiving unit 208 may directly receive the user attributes 222 from the user device 102.

In some embodiments, the attribute receiving unit 208 may be configured to receive the test results of a home-test kit, the results of a standardized health test administered by a medical professional, the results of a self-assessment tool used by the user, and/or the results of any external or third party test. Based on the results from any of these tests or tools, the attribute receiving unit 208 may be configured to determine the user attributes 222.

The recommendation system 204 may be configured to compare the plurality of user attributes 222 to a corresponding plurality of evidence-based nutritionally healthy benchmarks 228.

Furthermore, the attribute comparison unit 210 may be further configured to determine a benchmark set 232 based on the user's segment 230. For example, if the attribute comparison unit 210 determines that a user falls into the obese BMI segment 230, based on the plurality of user attributes 222, the attribute comparison unit 210 may select a benchmark set 232 that has been created and defined according to the specific needs for nutritional health before, during and/or after an IF diet (preferably a TRF regimen or an ADF regimen).

The comparison unit 210 may be further configured to select, from this determined nutritional benchmark set 232, the evidence-based nutritional benchmarks 128 and compare the now selected evidence-based nutritional benchmarks 228 to each of the corresponding user attributes 222. For example, when the nutritional benchmark set 232 has been determined, in response to the determination, the attribute comparison unit 210 may compare a user attribute 222 that represents the user's vitamin K intake to an evidence based nutritional benchmark 228 that represents a benchmark vitamin K intake, determining whether the user is below, at, or above the benchmark vitamin K intake. Though this example is based on a concrete, numerical comparison, another example of a benchmark comparison may be qualitative and different depending on a person. For example, a user attribute 222 may indicate that the user is currently experiencing higher than normal levels of stress. An example benchmark related to a user stress level may indicate that an average or low level of stress is desired and thus, the user attribute 222 indicating a higher level of stress is determined to be below that of the benchmark. As different users experience differing levels of stress, even under the same circumstances, such a comparison requires a customized approach.

During the comparison from the prior example, the attribute comparison unit 210 may be configured to determine a user nutritional score 234 based on the comparison between the evidence-based nutritional benchmarks 228 and the user attributes 222. For example, the attribute comparison unit 210 may determine a user nutritional score of 95/100 if the user attributes 222 very nearly meet all or most of the corresponding evidence-based nutritional benchmarks 228. In another example, a score may be represented through lettering grades, symbols, or any other system of ranking, for example “high,”, “average” or “low,” that allows a user to interpret how well their current attributes rate amongst benchmarks. This user nutritional score 234 may be presented through the display 206.

The recommendation system 204 may be further configured to determine a plurality of nutritional support opportunities 238 based on the plurality of user attributes 222 and the comparison to the corresponding plurality of evidence-based nutritional benchmarks 228. For example, the attribute comparison unit 210 may determine nutritional support opportunities 238 for every user attribute 222 that does not meet the corresponding evidence-based nutritional benchmark. In this example, a corresponding evidence-based nutritional benchmark 228 may require a user have an intake of 2 ug/day of folate, whereas the user attribute may indicate the user is only receiving 1 ug/day of folate. Therefore, the attribute comparison unit 210 may determine an increase in folate intake to be a nutritional support opportunity 238.

In another example, the attribute comparison unit 210 may be configured to identify a first set of user attributes 236 comprised of each of the plurality of user attributes 222 that are below the corresponding one of the plurality of evidence-based nutritional benchmarks 228 as well as identify a second set of user attributes 236 comprised of each of the plurality of user attributes 222 that are greater than or equal to the corresponding evidence-based nutritional benchmarks 228. While the first set of user attributes 236 is determined similarly to the above given example, the second set of user attributes 236 differs in that, although the associated user does not appear to have a deficiency, there may be opportunities to support nutritional health by recommending the user maintain current practices or opportunities to further improve upon them. Accordingly, the recommendation system 204 may determine opportunities to support nutritional health based on which attributes 222 populate either sets 236.

The recommendation system 204 may be further configured to identify a plurality of nutritional recommendations 240 based on the plurality of nutritional support opportunities 238. For example, the evidence-based diet and lifestyle recommendation engine 212 may be configured to be cloud-based. The recommendation engine 212 may comprise one or more of a plurality of databases 242, a plurality of dietary restriction filters 244, and an optimization unit 246. Based on the plurality of opportunities 238, the recommendation engine 212 may identify the plurality of nutritional recommendations 240 according to the one or more of plurality of databases 242, the dietary restriction filters 244, and the optimization unit 246.

In another example, the recommendation system 204 may be configured to provide continuous recommendations, based on prior user attributes. For example, the recommendation system 204 may comprise, in addition to the previously discussed elements, an attribute storing unit 216 and an attribute analysis unit 214. The attribute storing unit 216 may be configured to, responsive to the attribute receiving unit 108 receiving the plurality of user attributes 222, add the received user attributes 222 to an attribute history database 248 as a new entry based on when the plurality of user attributes 222 were received. For example, if user attributes 222 are received by the attribute receiving unit 208 on a first day, the attribute storing unit 216 will add the received user attributes 222 to a cumulative attribute history database 248 noting the date of entry, in this case the first day. Later, if user attributes 222 are received by the attribute receiving unit 208 on a second day, e.g. the next day, the attribute storing unit 216 will also add these new attributes to the attribute history database 248, noting that they were received on the second day, while also preserving the earlier attributes from the first day.

This attribute analysis unit 214 may be configured to analyze the plurality of user attributes 222 stored within the attribute history database 248, wherein analyzing the stored plurality of user attributes 222 comprises performing a longitudinal study 250. Continuing the earlier example, the attribute analysis unit 214 may perform a longitudinal study of the user attributes 222 from each of the first day, the second day, and every other collection of user attributes 222 found within the attribute history database 248. The evidence based diet and lifestyle recommendation engine 212 may be further configured to generate a plurality of nutritional recommendations 240 based on at least the stored user attributes 222 found within the attribute history database 248 and the analysis performed by the attribute analysis unit 214.

In an embodiment, the attribute analysis unit 214 is further configured to repeatedly analyze the plurality of user attributes 222 stored within the attribute history database 248 responsive to the attribute storing unit 216 adding a new entry to the attribute history database 248, essentially re-analyzing all of the data within the attribute history database 248 immediately after new user attributes 222 are received. Similarly, the evidence based diet and lifestyle recommendation engine 212 may be further configured to repeatedly generate the plurality of nutritional recommendations 240 responsive to the attribute analysis unit 214 completing an analysis, thereby effectively generating new nutritional recommendations 240 that consider all past and present user attributes 222 each time a new set of user attributes 222 is received.

In various embodiments, the user-specific (or population-specific) inputs to the disclosed system are programmable and configurable, and include gender, age, weight, height, physical activity level, whether obese, and the like. For example, FIG. 2 shows typical individual user data.

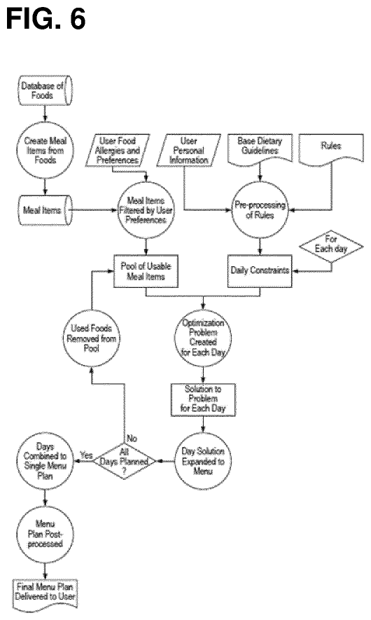

In an embodiment, the disclosed system includes or is connected to a database containing foods items, menus or recipes and respective nutrient content. In this embodiment, the disclosed system includes a fuzzy search feature that enables a user to enter a consumed (or to-be consumed) food, and thereafter searches the database to find a closest item to the user-provided item. The disclosed system, in this embodiment, uses stored nutritional information about the matched food item to determine whether it is a nutritionally-healthy item. For example, FIG. 6 shows an example of the workflow for a nutritionally-healthy menu plan.

In various embodiments, the disclosed system further includes an interface (e.g., a graphical user interface) to display the amount of each nutrient available in each food composing the diet, and displays the amount of energy available to be consumed.

In some embodiments, the interface enables users to modify the amount of various foods or energy to be consumed. In other embodiments, the system is configured to determine amounts of food or energy consumed using non-user-input data, such as by scanning one or more bar codes, QR codes, or RFID tags, image recognition systems, or by tracking items ordered from a menu or purchased at a grocery store.

Various embodiments of the disclosed system display a dashboard or other appropriate user interface to a user that is customized based on the user's needs. In embodiments of the system disclosed herein, a graphical user interface is provided which advantageously enables, for the first time, users to input data about food consumed in a given period of time and to see an indication of a score, based appropriately on energy consumption, that reflects overall nutritional content of the consume diet.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

In a preferred embodiment, the nutritional recommendations 240 (e.g., provided by the recommendation system 150 and/or the recommendation system 204) mitigate inadequate nutrient intake of an intermittent fasting (IF) diet, for example, a time-restricted feeding regimen (TRF). In a particularly preferred embodiment, the individual is administered and/or consumes nutrients according to the nutritional recommendations 240, for example calcium, preferably one or more calcium salts, such as calcium acetate, calcium carbonate, calcium chloride, calcium citrate, calcium gluconate, calcium lactate or mixtures thereof; and/or a fiber supplement, preferably those containing one or more of wheat dextrin, methylcellulose, psyllium husk, polycarbophil, guar fiber, glucomannan or β-glucans.

In view of the disclosures herein, an embodiment is a computer-implemented method for providing nutritional recommendations to an individual before, during and/or after an intermittent fasting (IF) diet, preferably a time-restricted feeding (TRF) regimen or an alternate day fasting (ADF) regimen.

Another embodiment is a computer-implemented system comprising: a menu database module; a recipe database module; a dietary constraints module; a nutrient scoring module; and an optimisation module, wherein the computer-implemented system is configured to generate nutritionally-healthy recommendations for an individual before, during and/or after an intermittent fasting (IF) diet, preferably a time-restricted feeding (TRF) regimen or an alternate day fasting (ADF) regimen.

In some embodiments, the nutritional recommendations comprise diet recommendations, menu recommendations and recipe recommendations.

Preferably, the nutritional recommendations comprise personalized nutritionally-healthy recommendations based on at least one individual parameter selected from the group consisting of age, gender, height, weight, BMI, medical condition, physical activity level, and total recommended daily energy allowance.

In some embodiments, the nutritional recommendations comprise individualized nutritionally-healthy recommendations based on at least one dietary preference or constraint selected from the group consisting of omnivorous diet, gluten-free, lactose-free, Mediterranean, paleo, vegan, and vegetarian.

Preferably, the nutritional recommendations identify at least one of a food comprising fiber, a supplement comprising fiber, an amount of a food or supplement comprising fiber, a food comprising calcium, a supplement comprising calcium, or an amount of a food or supplement comprising calcium.

In some embodiments, the nutritionally-healthy recommendations are selected from the group comprising food recommendations, menu recommendations and recipe recommendations for before, during and/or after an intermittent fasting (IF) diet, preferably a time-restricted feeding (TRF) regimen or an alternate day fasting (ADF) regimen.

Preferably, the nutritional recommendations comprise nutritionally-healthy recommendations for menus classified as suitable for a breakfast, a lunch, a dinner or a snack.

In some embodiments, the nutritional recommendations comprise recommendations of menus, wherein the menus are tagged as nutritionally-healthy for the user.

Preferably, the nutritional recommendations comprise recommendations of recipes, wherein the recipes are tagged as nutritionally-healthy for the user.

In another embodiment, a method of minimizing or preventing nutrient inadequacy in an individual performing an IF regimen is provided. The method comprises using any computer-implemented system disclosed herein to obtain the nutritionally-healthy recommendations; and administering to the individual one or more of a nutrient, a food containing a nutrient, or an amount thereof, according to the nutritionally-healthy recommendations from the computer-implemented system, the administration being performed to the individual before, during and/or after the IF diet. Preferably, at least one of a food comprising fiber, a supplement comprising fiber, an amount of a food or supplement comprising fiber, a food comprising calcium, a supplement comprising calcium, or an amount of a food or supplement comprising calcium is administered to the individual.

### Exemplary Menu Plan Building Workflow.

Each of the embodiments disclosed herein can build a menu plan using specific rules, constraints and goals in optimising the menu plan each day and over weeks based on the IF-healthy foods in order to create the recommendations. An example workflow for building the menu plan follows hereafter.

For a specific nutrient or ingredient, an optimal range can be specified by a lower and upper limit which define the possible range for the nutrient, as well as weighting for the lower and upper deviation from the ideal range. The rules include additional information such as units and whether the rule scaled with the amount of food.

Each goal is for a specific nutrient and includes a coefficient, used as a weight in the formula which was optimized, as well as whether the nutrient should be minimized or maximized.

### Rules for a Nutrient or Ingredient:

For each rule, r, corresponding to a nutrient or ingredient:
rₗₕᵣ=destred lower bound of rule
rᵤₕᵣ=destred upper bound of rule
rₗₕᵣ₀=absolute minimum for rule
rᵤₕᵣ₀=absolute maximum for rule
rₗₛ—weight for lower bound of rule
rᵤₛ=weight for upper bound of rule

These are specified goals, g, to either maximize or minimize the amount of nutrients and/or ingredients in the overall menu plan:
g_{coeff}=coefficient for goal g
g_{obj}∈{min,max}=whether g should be maximized or minimized

### Variables:

A database of meal items includes pre-created meals, m, which are suitable for use in menu plans. Each meal contained one or more foods in specified amounts, as well as summary nutrition information for the entire meal.

Each meal is represented by the variable f.

A variable theta for each meal, Θ, represents whether the meal will be included in the menu plan. It has nutritional information for each meal for all the specified rules and goals:
θ_{f}, f∈meals, θ_{f}∈{0,1}

A tag variable, t, indicates whether the meal is tagged for each occasion (breakfast, lunch, dinner, snacks.). These tags are used to ensure the correct number of meals each day.
m=meal occasions {breakfast,lunch,dinner,snack,etc.}
t_{f,m}∈{0,1} f∈meats, m∈occasions

### Constraints:

For each constraint, c, a constraint is placed upon the solution to the optimization problem.
mₗ=fewest number of meal occasion m to include
mᵤ=most number of meal occasion m to include
mₗ≤cₘ≤mᵤ, for m∈{breakfast,lunch,dinner,snack,etc.}

$${c}_{m} = {\sum }_{f ∈ meals} {t}_{f} {θ}_{f}$$

Each rule has two constraints with upper and lower bounds:
c_{l,r}=constraint, rₗₕᵣ≤c_{l,r}≤∞, for r∈rules
c_{u,r}=constraint, 0≤c_{l,r}≤rᵤₕᵣ, for r∈rules

For each rule, a slack variable, s, represents the deviation of the menu plan from the desired range for that nutrient:
s_{l,r}=lower end stack variable for rule r
s_{u,r}=upper end stack variable for rule r
0≤s_{l,r}≤rₗₕᵣ−rₗₕᵣ₀, for r∈rules
0≤s_{u,r}≤rᵤₕᵣ₀−rᵤₕᵣ, for r∈rules

Each constraint is the sum of the amount of each food to be included in the menu plan multiplied by the amount of that nutrient in the food, plus the corresponding slack variable:
c_{l,r}=lower end constraint for rule r
c_{u,r}=upper end constraint for rule r

$$\begin{matrix}{c}_{l , r} = {s}_{l , r} + {\sum }_{f ∈ meals} {0}_{f} \cdot {f}_{r} , & r ∈ rules \\ {c}_{u , r} = {s}_{u , r} + {\sum }_{f ∈ meals} {θ}_{f} * {f}_{r} , & r ∈ rules\end{matrix}$$

where fᵣ=amount of nutrient r in food f

### Objective Term:

An objective term, obj, for the goals is created by summing over each goal and each food, the amount of the food to be included in the menu plan plus the amount of the nutrient in the food multiplied by the goal coefficient, normalized by the maximum amount of the nutrient in all of the possible foods.

$$obj = {\sum }_{g ∈ goals} {\sum }_{f ∈ meals} {θ}_{f} + \frac{{f}_{g} \cdot {g}_{coeff}}{max \{ {food}_{g} \}}$$

where f_{g}=amount of nutrient g in food f

The menu plan is created by minimizing the sum of the slack variables over the rules weighted by the corresponding weight of the rule, plus the objective term described above.

$${min}_{θ} = {\sum }_{r ∈ rules} {s}_{l , r} * {r}_{ls} + {\sum }_{r ∈ rules} {s}_{u , r} * {r}_{us} + obj$$

The slack variables s measure the deviation of the resulting menu plan from the bounds for each nutrient specified in the rules. By minimizing the slack variables, the menu plan was able to respect the set rules. However, by using slack variables instead of specifying constraints allowed the menu plan greater leeway and recognized that it may not always be possible to exactly follow the rules.

The problem is solved by the optimization engine to produce the recommendations and resulting menu plans per day, week or month.

### EXAMPLES

The following non-limiting examples generally illustrate the concepts underlying the embodiments disclosed herein.

### Example 1: Analysis of Nutritional Inadequacies from National Health and Nutrition Examination Survey (NHANES) US Epidemiological Data

The National Health and Nutrition Examination Survey (NHANES) is a program of studies designed to assess the health and nutritional status of adults and children in the United States. The survey is unique in that it combines interviews and physical examinations. The NHANES interview includes demographic, socioeconomic, dietary, and health-related questions. The examination component consists of medical, dental, and physiological measurements, as well as laboratory tests administered by highly trained medical personnel. The sample for the survey is selected to represent the U.S. population of all ages. To produce reliable statistics, NHANES over-samples persons 60 and older, African Americans, and Hispanics. The cycles of data collection started in 1999 and it continues up to the date.

For this example, the NHANES cycles 2013-2014, 2015-2016, 2017-2018 were used, more specifically, the files “Dietary Interview-Individual Foods, First Day”, and “Demographic Variables and Sample Weights”. Datasets were downloaded from https://wwwn.cdc.gov/nchs/nhanes/default.aspx.

The dietary data consists of 1 or 2 24 h recalls, performed with the Automated Multiple-Pass Method. We analyzed only the first 24 h recall of each subject. From the dietary file, we extracted the list of foods reported by each individual, the time in HH:MM:SS, the name of the meal and the KCAL. This was merged with the demographics file to retrieve the age. A filter ≥18 was applied to the age. Eating/drinking occasions providing no calories (e.g. water, black coffee without sugar) were not considered, so that a single glass of water was not considered as an ‘eating occasion.’

### Calculation

For each subject, the time intervals between consecutive meals were calculated. For example, if meals were reported at 7:00, 12:00, 16:00, 20:00, then the time intervals are: 7 h, 5 h, 4 h, 4 h, 4 h.

### Descriptive Statistics

First and last time of eating. The number of subjects having reported food intake before 5 AM was n=1206.

Time-span of eating. For each subject, we report the time interval ‘Last time of eating—First time of eating.’

Counts of fasting intervals. For the rest of the analysis, we exclude subjects with first eating time before 5 AM. The interval between consecutive meals ranges from a few minutes to 23.5 hours, with a median of 3.5 hours.

The fasting intervals chosen for the analysis were as follows:
<12 hours fasting (n=64940)
12 hours to 16 hours fasting (n=4717)
>16 hours fasting (n=761). People in this group have reported eating times with a ‘gap’ of at least 16 h, for example no food consumption between 6 AM and 10 PM.

A cut-off of 5000 kcal was applied afterwards. All energy intakes above the cut-off were removed from the analysis. The analysis was repeated after excluding respondents who declared to have restricted their food intake because of insufficient money. It should be noted that the rate of non-response to the Food Security Questionnaire is very high (>50%).

The number of people within the different fasting intervals, and after applying the Food Security Questionnaire filter were as follows:
<12 hours fasting (n=14088),
12 hours to 16 hours fasting (n=1439), and
>16 hours fasting (n=129).

The following question from the Food Security Questionnaire was used to identify people restricting their food intake due to socioeconomical status:
“In the last 12 months, since last month, did {you/you or other adults in your household} ever cut the size of your meals or skip meals because there wasn't enough money for food?”
1: yes
2: No
9: Don't know

In NHANES, there are no questions related to intentional fasting, so it is not possible to make a direct connection between these intervals and a TRF regime. However, we assumed that the nutrient needs of people restricting eating to an 8-h interval would be similar to those intentionally following a 16:8 TRF diet.

### Time-Restricted Feeding Intervals Analysis

Macronutrient intakes were calculated for the three chosen fasting intervals, <12 h, 12 h to 16 h, and >16 h (Table 1). The repartition of energy among fats, proteins and carbohydrates is similar between the three groups; means and medians fall within the acceptable macronutrient distribution ranges: Fats (20-35% of energy), Protein (10-35% of energy) and Carbohydrate (45-65% of energy), as defined by the US National Academy of Sciences (https://ods.od.nih.gov/HealthInformation/Dietary_Reference_Intakes.aspx).

TABLE 1
Macronutrient intakes as a percentage of energy, by fasting interval.

| TRF | Fat intake | Protein intake | Carbohydrate intake |
|---|---|---|---|
| period | % Daily Energy | % Daily Energy | % Daily Energy |
| <12 h | Mean (SD): 33.6 (9.29) | Mean (SD): 15.8 (5.59) | Mean (SD): 49.5 (11.5) |
| (N = 1858) | Median: 34.0 | Median: 14.9 | Median: 49.6 |
| | Min, Max: 2.76, 66.2 | Min, Max: 1.81, 50.3 | Min, Max: 12.2, 91.8 |
| 12 h-16 h | Mean (SD): 33.6 (9.95) | Mean (SD): 15.6 (6.04) | Mean (SD): 48.3 (11.4) |
| (N = 250) | Median: 34.8 | Median: 14.8 | Median: 47.4 |
| | Min, Max: 1.51, 58.8 | Min, Max: 0.401, 40.5 | Min, Max: 19.7, 89.1 |
| >16 h | Mean (SD): 34.3 (10.1) | Mean (SD): 16.5 (7.59) | Mean (SD): 47.9 (12.9) |
| (N = 48) | Median: 35.8 | Median: 14.8 | Median: 48.2 |
| | Min, Max: 6.10, 55.9 | Min, Max: 7.46, 46.9 | Min, Max: 12.1, 79.8 |
| Overall | Mean (SD): 33.6 (9.39) | Mean (SD): 15.8 (5.69) | Mean (SD): 49.3 (11.5) |
| (N = 2156) | Median: 34.0 | Median: 14.9 | Median: 49.4 |
| | Min, Max: 1.51, 66.2 | Min, Max: 0.401, 50.3 | Min, Max: 12.1, 91.8 |

Micronutrient intakes were calculated for the three chosen fasting intervals, <12 h, 12 h to 16 h, and >16 h (Table 2). All the nutrients considered have statistically significant distributions between the groups “<12 h” and “>16 h” (Wilcoxon test, p<0.05), even after adjusting for energy cofounders (adjusted by 1000 kCal).

TABLE 2
Distributions of micronutrient intakes per 1000 kcal, by fasting interval

| Nutrient | Group | Minimum | Quartile1 | Mean | Median | Quartile3 | Max |
|---|---|---|---|---|---|---|---|
| Calcium | <12 h | 41.2 | 249.5 | 405.3 | 371.6 | 521.5 | 1675.4 |
| | 12 h-16 h | 37.5 | 196.1 | 345.0 | 315.7 | 450.8 | 1042.5 |
| | >16 h | 49.6 | 139.3 | 338.2 | 281.8 | 473.3 | 1140.6 |
| Fiber | <12 h | 0.1 | 5.0 | 8.4 | 7.3 | 10.6 | 66.9 |
| | 12 h-16 h | 0.7 | 4.5 | 7.4 | 6.2 | 8.9 | 39.0 |
| | >16 h | 1.7 | 4.2 | 7.2 | 5.7 | 8.0 | 22.7 |
| Folate | <12 h | 12.6 | 125.9 | 194.9 | 169.2 | 235.8 | 3128.3 |
| | 12 h-16 h | 24.3 | 88.2 | 158.2 | 132.5 | 199.6 | 415.6 |
| | >16 h | 0.0 | 107.1 | 164.9 | 147.5 | 205.9 | 834.5 |
| Iron | <12 h | 1.1 | 4.9 | 7.0 | 6.2 | 7.9 | 46.6 |
| | 12 h-16 h | 1.3 | 4.4 | 6.2 | 5.8 | 7.3 | 19.8 |
| | >16 h | 2.1 | 4.2 | 6.0 | 5.9 | 7.1 | 12.5 |
| Magnesium | <12 h | 13.4 | 98.3 | 133.3 | 124.0 | 156.4 | 995.0 |
| | 12 h-16 h | 30.4 | 87.2 | 119.5 | 108.9 | 145.0 | 347.4 |
| | >16 h | 53.9 | 82.6 | 118.4 | 97.4 | 127.8 | 359.8 |
| Niacin | <12 h | 0.9 | 8.7 | 12.4 | 11.2 | 14.5 | 59.8 |
| | 12 h-16 h | 0.5 | 8.8 | 12.8 | 11.4 | 15.3 | 89.3 |
| | >16 h | 4.2 | 8.6 | 13.6 | 11.8 | 16.1 | 57.6 |
| Potassium | <12 h | 144.7 | 938.6 | 1254.1 | 1185.6 | 1484.9 | 4269.9 |
| | 12 h-16 h | 297.2 | 832.9 | 1130.1 | 1065.0 | 1325.1 | 2612.6 |
| | >16 h | 389.7 | 801.7 | 1176.0 | 942.7 | 1352.4 | 2997.3 |
| Selenium | <12 h | 7.3 | 41.6 | 56.6 | 52.7 | 67.1 | 300.9 |
| | 12 h-16 h | 0.8 | 40.1 | 54.9 | 52.7 | 67.0 | 158.2 |
| | >16 h | 4.6 | 36.9 | 58.3 | 52.0 | 69.6 | 226.7 |
| Sodium | <12 h | 315.6 | 1309.5 | 1676.7 | 1599.5 | 1961.7 | 13041.6 |
| | 12 h-16 h | 82.6 | 1331.1 | 1688.0 | 1596.5 | 1940.8 | 4847.5 |
| | >16 h | 996.0 | 1361.5 | 1728.4 | 1649.7 | 2031.5 | 3339.5 |
| Vitamin A | <12 h | 0.0 | 124.8 | 284.6 | 218.5 | 345.5 | 4039.7 |
| | 12 h-16 h | 0.0 | 87.0 | 240.3 | 162.1 | 282.9 | 2931.9 |
| | >16 h | 6.2 | 73.0 | 228.9 | 148.7 | 252.5 | 1537.7 |
| Thiamin | <12 h | 0.1 | 0.6 | 0.8 | 0.7 | 0.9 | 5.4 |
| | 12 h-16 h | 0.0 | 0.5 | 0.7 | 0.6 | 0.8 | 3.0 |
| | >16 h | 0.2 | 0.5 | 0.7 | 0.6 | 0.8 | 1.4 |
| Vitamin B12 | <12 h | 0.0 | 1.1 | 2.3 | 1.7 | 2.8 | 45.8 |
| | 12 h-16 h | 0.0 | 0.9 | 2.1 | 1.5 | 2.7 | 24.6 |
| | >16 h | 0.0 | 0.7 | 1.8 | 1.4 | 1.9 | 12.7 |
| Riboflavin | <12 h | 0.2 | 0.7 | 1.0 | 0.9 | 1.1 | 7.6 |
| | 12 h-16 h | 0.1 | 0.5 | 0.9 | 0.7 | 1.0 | 13.1 |
| | >16 h | 0.1 | 0.6 | 0.8 | 0.7 | 0.8 | 4.6 |
| Vitamin B6 | <12 h | 0.1 | 0.6 | 1.0 | 0.9 | 1.2 | 27.4 |
| | 12 h-16 h | 0.0 | 0.6 | 1.0 | 0.8 | 1.2 | 8.8 |
| | >16 h | 0.3 | 0.5 | 1.0 | 0.8 | 1.3 | 5.5 |
| Vitamin C | <12 h | 0.0 | 10.1 | 41.1 | 24.1 | 55.4 | 621.3 |
| | 12 h-16 h | 0.0 | 6.3 | 39.0 | 17.6 | 49.2 | 323.0 |
| | >16 h | 0.2 | 4.6 | 39.4 | 12.9 | 42.2 | 369.9 |
| Vitamin D | <12 h | 0.0 | 0.6 | 2.3 | 1.7 | 3.0 | 31.9 |
| | 12 h-16 h | 0.0 | 0.3 | 1.7 | 0.8 | 2.1 | 26.9 |
| | >16 h | 0.0 | 0.2 | 1.6 | 0.6 | 1.3 | 21.3 |
| Vitamin E | <12 h | 0.1 | 2.5 | 3.9 | 3.5 | 4.6 | 37.1 |
| | >16 h | 1.2 | 2.5 | 3.9 | 3.6 | 4.2 | 12.5 |
| | 12 h-16 h | 0.1 | 2.5 | 3.7 | 3.4 | 4.5 | 16.2 |
| Zinc | <12 h | 0.8 | 3.7 | 5.2 | 4.7 | 6.0 | 70.0 |
| | 12 h-16 h | 0.7 | 3.4 | 5.0 | 4.5 | 5.7 | 15.2 |
| | >16 h | 1.3 | 3.1 | 4.4 | 4.0 | 5.1 | 14.2 |

### Risk of Inadequate Intakes of Time Restricted Feeding, by Fasting Intervals

| | Percent Below Recommendation* | | |
|---|---|---|---|
| | <12 h fasting | 12 h to 16 h fasting | >16 h fasting |
| Nutrient | N = 1858 | N = 250 | N = 48 |
| Calcium | 45.4 | 64.4 | 66.7 |
| Fiber | 90.2 | 95.2 | 97.9 |
| Folate | 47.3 | 61.2 | 81.2 |
| Iron | 14.7 | 26.8 | 45.8 |
| Magnesium | 65.6 | 79.2 | 89.6 |
| Potassium | 95.7 | 98.0 | 97.9 |
| Vitamin A | 63.0 | 77.6 | 81.2 |
| Vitamin B12 | 25.7 | 38.0 | 54.2 |
| Vitamin C | 60.8 | 68.4 | 79.2 |
| Vitamin D | 90.5 | 94.4 | 97.9 |
| Vitamin E | 83.8 | 87.6 | 95.8 |
| Zinc | 38.4 | 50.0 | 72.9 |

*Percent below the Estimated Average Requirement, except for potassium and fiber, where the percent below the Adequate Intake is shown.

### Example 2: Risk of Inadequate Nutrient Intakes of Intermittent Fasting Protocols with Simulated Diets

In order to estimate the potential risk of nutrients inadequacies for TRF diet, without the use of randomized controlled trials (RCT) or traditional dietary assessment methods (e.g., 24-hours food recall, food frequency questionnaire, food records), we simulated approximately 20,000 days of food intake in silico for a variety of individuals adhering to an TRF diet with a digital tool. For this example, 16:8 protocol was used as an illustrative example of a TRF diet.

The digital simulation tool simulates multiple days of food intake by finding the optimal combination of available meals to maximize the nutritional balance of the meals by conforming as best as possible to the USDA Healthy Eating Plan guidelines. In order to mimic real-world food intake as closely as possible, we used actual meals consumed by people as reported in the National Health and Nutrition Examination Survey (NHANES), a survey performed by the US Center for Disease Control and Prevention (CDC). Datasets were downloaded from https://wwwn.cdc.gov/nchs/nhanes/default.aspx. The NHANES cycles 2013-2014, 2015-2016, 2017-2018 were used, more specifically, the files “Dietary Interview-Individual Foods, First Day,” and “Demographic Variables and Sample Weights.”

This allows for the simulated diets to consist of meals actually consumed by the US population.

It is important to note that specific nutritional inadequacies will depend on dietary habits and cultural characteristics and will likely differ in different populations around the world. The system or method described here will be the same, but the nutritional inadequacies identified may differ.

Within the simulation tool, the user specifies the characteristics for a set of individuals based on sex, age, height, weight and physical activity level in order to calculate the estimated energy requirement (EER) per day. The simulation tool then uses integer programming techniques to create in silico menu plans which optimize the nutritional content of the overall diet. In this Example, the simulation tools rely on food group level diet recommendations as specified by the USDA Healthy Eating Patterns which provide target amounts of food groups to be consumed on a daily and weekly basis. (The simulation tool can be adapted to different food-based dietary guidelines if applied in other geographies.)

Additional nutrient level constraints are set as upper limits on nutrients such as sodium, added sugars, and saturated fats. The constraints are adjusted based on the individual's EER to obtain desired ranges of food groups and nutrients to be consumed per day and per week. An objective function is created as a scalar weighted linear combination of the differences between the actual amount of each individual food group and nutrient and the optimal range. The diet is created by selecting the combination of meals which meet the energy requirements while attempting to keep the consumption of food groups and nutrients within the optimal range.

### Baseline Diet Simulation

To simulate the dietary intakes of TRF regimen (e.g., 16:8), we simulated approximately 20,000 days of “baseline” diet by maximizing the Healthy Eating Patterns as described above with no other rules applied. This “baseline” diet is considered to be a healthy and balanced diet (Table 4). In this Example, the baselines were simulated for 1,400 individuals of each gender, ranging in age from 18 to 70, of varying heights and weights and sedentary physical activity level, for 7 days each. While the use of meals extracted from NHANES forces the simulated diets to use meals reportedly consumed by actual people, the optimization of the balance of food groups produces diets with a bias towards healthier diets than what people are likely to actually eat. As such we considered these to be “ideal” diets rather than realistic diets, which will tend to have higher nutrient intakes than occur in the real world.

TABLE 4
Percent of individuals with inadequate intakes
at baseline (from diet simulation models).

| | Males | | | Females | | |
|---|---|---|---|---|---|---|
| | Mean Intake | | Percent | Mean Intake | | Percent |
| Nutrient | Baseline | | Inadequate | Baseline | | Inadequate |
| Total fat | 47.1 | g | 0 | 41.7 | g | 0 |
| Fiber | 28.9 | g | 77 | 27.4 | g | 17 |
| Calcium | 1240.1 | mg | 9 | 1090.7 | mg | 41 |
| Copper | 1.7 | μg | 0 | 1.5 | μg | 0 |
| Food folate | 387.4 | μg | 12 | 376.0 | μg | 30 |
| Iron | 14.3 | mg | 0 | 12.2 | mg | 3 |
| Magnesium | 402.3 | mg | 2 | 370.8 | mg | 4 |
| Niacin | 20.9 | mg | 0 | 17.4 | mg | 7 |
| Potassium | 5121.9 | mg | 0 | 4620.0 | mg | 0 |
| Riboflavin | 2.3 | mg | 0 | 2.0 | mg | 0 |
| Selenium | 93.2 | μg | 0 | 77.4 | μg | 9 |
| Sodium | 2256.2 | mg | 1 | 1931.4 | mg | 24 |
| Vitamin A | 1076.1 | μg | 9 | 875.3 | μg | 23 |
| Vitamin B12 | 4.2 | μg | 2 | 3.3 | μg | 17 |
| Vitamin B6 | 3.0 | mg | 0 | 2.6 | mg | 1 |
| Vitamin C | 496.4 | mg | 0 | 479.5 | mg | 0 |
| Vitamin D | 7.5 | μg | 59 | 5.6 | μg | 86 |
| Vitamin E | 8.2 | mg | 94 | 8.0 | mg | 93 |
| Vitamin K | 113.7 | μg | 80 | 122.2 | μg | 52 |
| Zinc | 10.6 | mg | 18 | 9.0 | mg | 25 |

It is noted that some nutrients are low at baseline, meaning they are not at risk particularly because of IF diets, but in general in US diet/baseline.

### 16:8 TRF Protocol Diet Simulation

The 16:8 TRF protocol was created from the baseline diet by removing meals as per the rules of the protocol: 16 h fasting and 8 hours eating. In this case, the simulation was done by skipping breakfast meal occasion.

By comparing the baseline inadequacies to the different TRF protocols, we can identify how nutrient intakes change when following this type of TRF protocol (Table 5). This method of simulation, where a baseline diet is modified to comply with the rules of the IF protocol, forces the results to be comparable, allowing us to identify the relative risks of nutrient inadequacy for the TRF diet while controlling for randomness inherent in the simulation process and for nutrient inadequacies which may result from the simulation process rather than being inherent in the TRF regimen.

TABLE 5
Percent of individuals with inadequate intakes when rules for
16:8 TRF regimen were applied (from diet simulation models).

| | Percent of | Percent of | Percent of |
|---|---|---|---|
| | Inadequacy, | Inadequacy, | Inadequacy, both |
| Nutrient | Males | Females | Males and Females |
| Calcium | 29.0% | 55.0% | 42.0% |
| Copper | 0.0% | 3.0% | 1.0% |
| Fiber | 95.0% | 69.0% | 82.0% |
| Food Folate | 33.0% | 52.0% | 42.0% |
| Iron | 0.0% | 13.0% | 7.0% |
| Magnesium | 41.0% | 28.0% | 35.0% |
| Niacin | 1.0% | 14.0% | 8.0% |
| Potassium | 1.0% | 1.0% | 1.0% |
| Riboflavin | 4.0% | 8.0% | 6.0% |
| Selenium | 0.0% | 15.0% | 8.0% |
| Sodium | 2.0% | 34.0% | 18.0% |
| Vitamin A | 18.0% | 30.0% | 24.0% |
| Vitamin B12 | 17.0% | 43.0% | 30.0% |
| Vitamin B6 | 0.0% | 9.0% | 5.0% |
| Vitamin C | 0.0% | 0.0% | 0.0% |
| Vitamin D | 85.0% | 93.0% | 89.0% |
| Vitamin E | 99.0% | 98.0% | 99.0% |
| Vitamin K | 84.0% | 58.0% | 72.0% |
| Zinc | 43.0% | 42.0% | 43.0% |

The diet for each simulated individual is analyzed by comparing the mean intakes of each nutrient to the DRI for that individual, based on their age and gender. As nutrient intakes tend to not be normally distributed, bootstrap confidence intervals are created for the mean which are compared to the appropriate DRI. In order to pool results for individuals with varying DRIs, the result for each individual is calculated as a ratio of their mean intake to their DRI. The overall gaps between simulated intake and DRI are created by taking the mean of these ratios. The risks of inadequacies are created by taking the percentage of simulated individuals with inadequate intake for each nutrient.

The results of the analyses and the resulting recommendations for each group are below. These tables show the complete results of the analysis.

### Risk of Inadequate Intakes of 16:8 TRF Regimen

Tables 6-7 show the risk for inadequacies identified for males and females for 16:8 TRF regimen. Most nutrients have an EAR, but when there were not sufficient data to assign an EAR, an AI was assigned instead (Institute of Medicine, US). In the Tables below, “Mean Inadequacy” is the amount below EAR or AI. The “Percent Inadequate” indicates the percent of individuals below the EAR or AI. If a large portion of the modelled diets produce inadequacies, then those nutrients are considered to be “At Risk” for inadequacies. Note that the interpretation is somewhat different for the AI; with a percent above an AI, we can generally assume adequacy, but a percent below does not necessarily imply inadequacy. A negative Mean Inadequacy indicates that the intakes tend to be adequate.

TABLE 6
Nutritional Analysis of 16:8 TRF for Males.

| | | | | Mean | Percent | |
|---|---|---|---|---|---|---|
| | | | EAR/AI | Inade- | Inade- | |
| Nutrient | Type | Units | Value | quacy | quate | Assessment |
| Fiber | AI | g | 34.8 | 10.9 | 94.8 | |
| Calcium | EAR | mg | 860 | −204.6 | 29.4 | |
| Copper | EAR | mcg | 0.7 | −0.8 | 00.0 | |
| Food Folate | EAR | mcg | 320 | −16.4 | 33.1 | At Risk |
| Iron | EAR | mg | 6 | −4.9 | 00.1 | |
| Magnesium | EAR | mg | 346 | −10.1 | 41.1 | At Risk |
| Niacin | EAR | mg | 12 | −7.1 | 1.4 | |
| Potassium | AI | mg | 3000 | −1665.7 | 0.6 | |
| Protein | EAR | g | 56 | −20.7 | 8.2 | |
| Riboflavin | EAR | mg | 1.1 | −0.8 | 3.7 | |
| Selenium | EAR | mcg | 45 | −50.6 | 0.4 | |
| Sodium | AI | mg | 1500 | −765.3 | 2.3 | |
| Vitamin A | EAR | mcg | 625 | −451.0 | 17.6 | |
| Vitamin B12 | EAR | mcg | 2.0 | −1.3 | 17.3 | |
| Vitamin B6 | EAR | mg | 1.22 | −1.4 | 0.0 | |
| Vitamin C | EAR | mg | 75 | −364.1 | 0.0 | |
| Vitamin D | EAR | mcg | 10 | 3.2 | 85.4 | |
| Vitamin E | EAR | mg | 12 | 5.1 | 98.7 | |
| Vitamin K | AI | mcg | 120 | 26.8 | 84.5 | |
| Zinc | EAR | mg | 9.40 | −0.3 | 43.4 | At Risk |

TABLE 7
Nutritional Analysis of 16:8 TRF for Females.

| | | | | Mean | Percent | |
|---|---|---|---|---|---|---|
| | | | EAR/AI | Inade- | Inade- | |
| Nutrient | Type | Units | Value | quacy | quate | Assessment |
| Fat total | EAR | g | 18.4 | −18.4 | 0.4 | |
| Fiber | AI | g | 23.4 | 2.1 | 68.9 | |
| Calcium | EAR | mg | 880 | 25.1 | 55.1 | At Risk |
| Copper | EAR | mcg | 0.7 | −0.6 | 2.9 | |
| Food Folate | EAR | mcg | 320 | 7.5 | 51.6 | At Risk |
| Iron | EAR | mg | 6.86 | −2.4 | 13.1 | |
| Magnesium | EAR | mg | 263 | −36.6 | 28.4 | |
| Niacin | EAR | mg | 11 | −3.2 | 14.1 | |
| Potassium | AI | mg | 2300 | −1414.4 | 1.1 | |
| Protein | EAR | g | 46 | −9.6 | 23.4 | |
| Riboflavin | EAR | mg | 0.9 | −0.6 | 8.4 | |
| Selenium | EAR | mcg | 45 | −23.2 | 15.3 | |
| Sodium | AI | mg | 1500 | −252.0 | 34.4 | |
| Vitamin A | EAR | mcg | 500 | −254.7 | 29.5 | |
| Vitamin B12 | EAR | mcg | 2.0 | −0.4 | 43.3 | At Risk |
| Vitamin B6 | EAR | mg | 1.18 | −0.8 | 9.1 | |
| Vitamin C | EAR | mg | 60 | −316.2 | 0.0 | |
| Vitamin D | EAR | mcg | 10 | 5.7 | 92.8 | |
| Vitamin E | EAR | mg | 12 | 5.3 | 98.4 | |
| Vitamin K | AI | mcg | 90 | −25.0 | 58.5 | |
| Zinc | EAR | mg | 6.8 | −0.5 | 41.8 | At Risk |

### Analysis: Distributions of Intakes

Tables 8-9 show the distributions of intakes for the nutrients of interest. The nutrients of interest are selected as those which have a risk of inadequacy large enough to be notable, and where the percentage of simulated diets which have inadequate intakes differs significantly from the baseline.

TABLE 8
Distribution of Intakes for Nutrients of Risk for 16:8 TRF regimen, Males.

| Nutrient | Units | EAR/AI | RDA | Minimum | 2.5% | 50% | 97.5% | Maximum |
|---|---|---|---|---|---|---|---|---|
| Fiber | g | 34.8 | NA | 8.1 | 14.5 | 22.7 | 37.3 | 51.5 |
| Calcium | mg | 860 | 1000 | 269.4 | 358.1 | 968.2 | 2162.6 | 2667.4 |
| Food Folate | mcg | 320 | 400 | 33.7 | 134.7 | 340.3 | 503.8 | 573.5 |
| Magnesium | mg | 346 | 416 | 178.9 | 260.4 | 354.9 | 439.5 | 490.8 |
| Vitamin D | mcg | 10 | 15 | 0.0 | 0.5 | 6.1 | 19.3 | 75.7 |
| Vitamin E | mg | 12 | 15 | 3.1 | 4.4 | 6.4 | 12.2 | 19.3 |
| Vitamin K | mcg | 120 | NA | 7.3 | 26.8 | 74.3 | 290.4 | 1781.3 |
| Zinc | mg | 9.4 | 11 | 3.3 | 4.6 | 9.7 | 16.2 | 31.8 |

TABLE 9
Distribution of Intakes for Nutrients of Risk for 16:8 TRF regimen, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 154.5 | 306.3 | 802.2 | 2125.7 | 2667.4 |
| Fiber | g | 23.4 | NA | 6.0 | 11.2 | 21.6 | 31.5 | 42.8 |
| Food Folate | mcg | 320 | 400 | 50.4 | 118.0 | 318.7 | 499.0 | 570.8 |
| Magnesium | mg | 263 | 318 | 127.8 | 173.3 | 303.8 | 403.8 | 514.5 |
| Potassium | mg | 2300 | NA | 1530.4 | 2237.0 | 3517.2 | 5669.2 | 6086.3 |
| Vitamin A | mcg | 500 | 700 | 1.8 | 119.2 | 702.7 | 1436.9 | 1776.7 |
| Vitamin B12 | mcg | 2.0 | 2.4 | 0.0 | 0.4 | 2.0 | 6.1 | 8.0 |
| Vitamin D | mcg | 10 | 15 | 0.0 | 0.1 | 3.7 | 14.7 | 20.5 |
| Vitamin K | mcg | 90 | NA | 5.2 | 19.0 | 80.1 | 331.4 | 582.1 |
| Zinc | mg | 6.8 | 8 | 2.1 | 3.5 | 7.4 | 11.3 | 17.0 |

### Analysis: Amount of Inadequacy

Tables 10-11 show the amount below the EAR or AI for each percentile (2.5%, 50% and 97.5%) for the nutrients of interest. The nutrients of interest are selected as those which have a risk of inadequacy large enough to be notable, and where the percentage of simulated diets which have inadequate intakes differs significantly from the baseline. If the values are negative it means that the intake tends to be above the DRI.

TABLE 10
Amount of inadequacy of each “At Risk”
nutrient for 16:8 TRF for Males.

| Nutrient | Units | EAR/AI | 2.5% Gap | 50% Gap | 97.5% Gap |
|---|---|---|---|---|---|
| Calcium | mg | 860 | 501.9 | −108.2 | −1302.6 |
| Fiber | g | 34.8 | 20.3 | 12.1 | −2.5 |
| Food Folate | mcg | 320 | 185.3 | −20.3 | −183.8 |
| Magnesium | mg | 346 | 85.6 | −8.9 | −93.5 |
| Vitamin D | mcg | 10 | 9.5 | 3.9 | −9.3 |
| Vitamin E | mg | 12 | 7.6 | 5.6 | −0.2 |
| Vitamin K | mcg | 120 | 93.2 | 45.7 | −170.4 |
| Zinc | mg | 9.4 | 4.8 | −0.3 | −6.8 |

TABLE 11
Amount of inadequacy of each “At Risk”
nutrient for 16:8 TRF for Females.

| Nutrient | Units | EAR/AI | 2.5% Gap | 50% Gap | 97.5% Gap |
|---|---|---|---|---|---|
| Calcium | mg | 880 | 573.7 | 77.8 | −1245.7 |
| Fiber | g | 23.4 | 12.2 | 1.8 | −8.1 |
| Food Folate | mcg | 320 | 202.0 | 1.3 | −179.0 |
| Magnesium | mg | 263 | 89.7 | −40.8 | −140.8 |
| Potassium | mg | 2300 | 63.0 | −1217.2 | −3369.2 |
| Vitamin A | mcg | 500 | 380.8 | −202.7 | −936.9 |
| Vitamin B12 | mcg | 2.0 | 1.6 | 0.0 | −4.1 |
| Vitamin D | mcg | 10 | 9.9 | 6.3 | −4.7 |
| Vitamin K | mcg | 90 | 71.1 | 10.0 | −241.4 |
| Zinc | mg | 6.8 | 3. | −0.6 | −4.5 |

The analysis of epidemiological data from NHANES database (Example 1) supports the fact the temporal eating patterns compatible with an intermittent fasting regimen are more likely to be associated with inadequate intakes of micronutrients. This suggest that the results produced by the diet simulator are consistent with patterns observed in the population. However, NHANES is essentially cross-sectional, while the diet simulator has a longitudinal dimensional that is difficult to measure in the population at a large scale.

### Recommendations (Applicable for Examples 1 and 2)

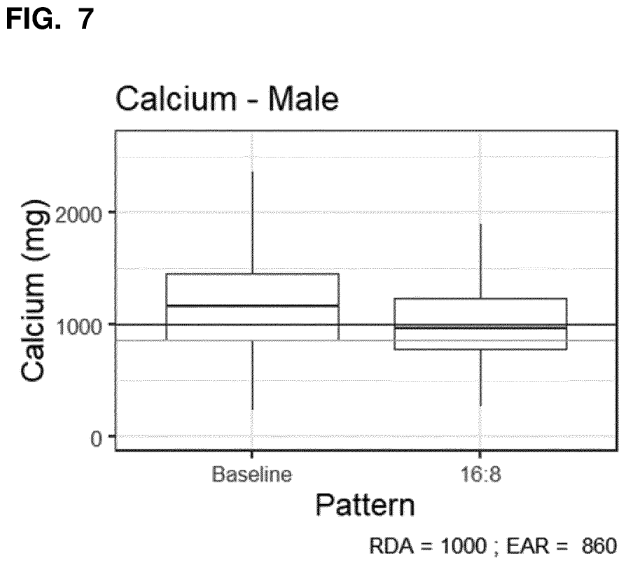

In order to construct dietary recommendations, we consider the upper and lower bounds of the intake recommendations to identify nutrient targets, without exceeding the UL (see FIGS. 7-10 for examples for calcium and fiber). The “Amount of Inadequacy” is used to generate the dietary recommendations. First, we take the difference between the RDA, or the AI if no RDA is available, and the 2.5% intake percentile as the upper and the difference with the 97.5% intake percentile as the lower. All recommendations are based on an energy level for the diet of 2000 kcal. The upper end of the recommendation is capped to ensure that the recommendation does not exceed the UL.

We then employ a computerized Diet Simulator. By making certain assumptions, the Diet Simulator can provide a reasonable estimate of the nutritional gaps and thus provide recommendations on how to close those gaps from nutrients, foods, beverages, and/or dietary supplements, and their combinations, without requiring undue effort or input from the user. In this Example, assumptions included the following:
The individual eats a typical meal pattern (e.g., breakfast, lunch, dinner and one snack per day),
The individual eats typical food and beverage combinations for those meals and snacks (e.g., meals and snacks as reported in NHANES), and
The individual intends to follow a well-balanced diet in accordance with dietary recommendations (e.g., the USDA Healthy Eating Pattern).

The food composition database used by the Diet Simulator contains the nutrient content, including macro- and micro-nutrients for each food per 100 g and per representative serving sizes. In this example, the USDA Food Data Central databases are used to identify foods and beverages containing sufficient levels of the nutrients identified as “At Risk” in the preceding steps. The Diet Simulator is also linked to a recipe database so that recipes which contain food sources of the nutrients “At Risk” can be identified.

In both methodologies, we identified inadequacies (>80% inadequacy) in vitamin D, vitamin E and fiber, both at baseline diet and TRF diet. In both methodologies, we also identified inadequacies (>40% inadequacy) that are exclusively related to the TRF diet: calcium, magnesium, iron, zinc, vitamin B12 and folate. Potassium and vitamin C inadequacies were also identified in one of the methodologies (Example 1). We have selected only two of these nutrients to exemplify recommendations.

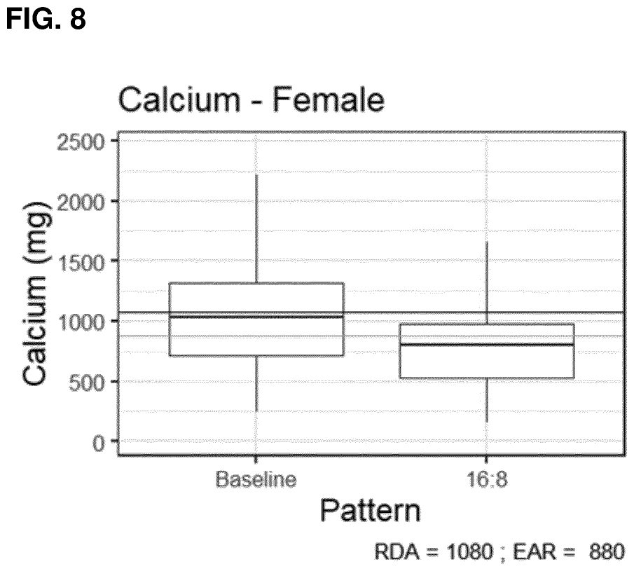

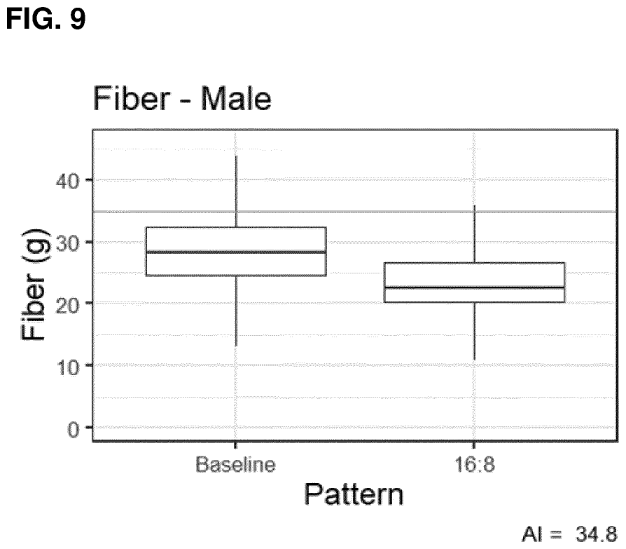

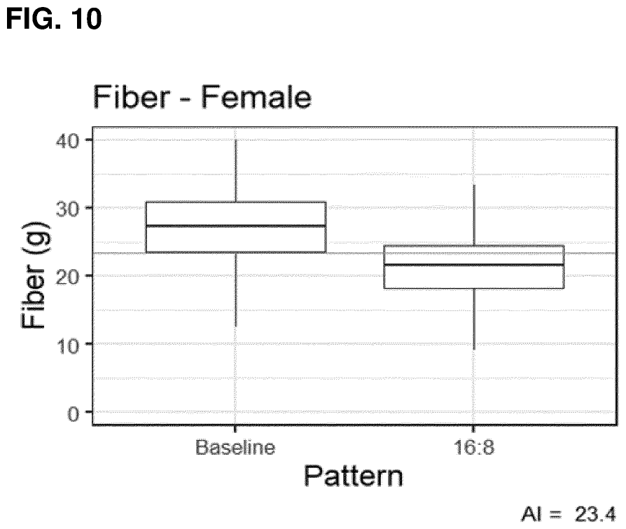

In these examples, fiber and calcium were identified as nutrients of need for the 16:8 TRF regimen. See FIG. 7 shows calcium intake ranges comparing simulated baseline intakes with 16:8 TRF regimen for males. FIG. 8 shows calcium intake ranges comparing simulated baseline intake with 16:8 TRF regimen for females. FIG. 9 shows fiber intake ranges comparing simulated baseline intakes with 16:8 TRF regimen for males. FIG. 10 shows fiber intake ranges comparing simulated baseline intakes with 16:8 TRF regimen for females.

Examples, such as the following dietary recommendations would be generated to accompany feedback on the TRF diets. In a similar manner, dietary recommendations for all of the nutrients identified as “At Risk” could be generated. As an illustration, we provide examples for calcium and dietary fiber as follows:

### Calcium:

Calcium could be recommended from food sources. For example, someone specifying a vegetarian diet may receive a recommendation for soybean curd (tofu) cubes; 1 cup (248 g) provides 275 mg of calcium. Similarly, a recipe for tofu with mixed vegetables, including broccoli and carrots with a soy-based sauce could be recommended; 2 cups (434 g) provides 286 mg of calcium.

Calcium could be recommended from beverages. For example, someone specifying a flexitarian diet may receive a recommendation for low-fat milk; 1 cup (246 g) of low-fat milk contains 310 mg of calcium.

Calcium could be recommended as a dietary supplement. Non-limiting examples of suitable forms of calcium include one or more calcium salts, such as calcium acetate, calcium carbonate, calcium chloride, calcium citrate, calcium gluconate, calcium lactate or mixtures thereof.

The amount of additional calcium recommended would depend on the needs identified in the simulations.

### Fiber:

Fiber could be recommended from food sources. For example, someone specifying a Vegan diet may receive a recommendation for cooked oatmeal; 1 cup (234 g) provides 4 mg of dietary fiber. Similarly, a recipe for oatmeal raisin cookies could be recommended; 2 cookies (48 g) provides 1.6 g of fiber.

Fiber could be recommended from dietary supplements. Non-limiting suitable forms of fiber supplements include those containing wheat dextrin, methylcellulose, psyllium husk, polycarbophil, guar fiber, glucomannan or β-glucans.

### Example 3: Risk of Inadequate Nutrient Intakes of Intermittent Fasting Protocols with Simulated Diets

In order to estimate the potential risk of nutrients inadequacies for ADF diets, without the use of randomized controlled trials (RCT) or traditional dietary assessment methods (e.g., 24-hours food recall, food frequency questionnaire, food records), we simulated approximately 20,000 days of food intake in silico for a variety of individuals adhering to an IF diet with the digital tool used in the preceding examples.

### Baseline Diet Simulation

To simulate the dietary intakes of different ADF regimens in a manner such that the results for the various protocols (Strict ADF, Modified ADF and 5:2) are comparable, we simulated approximately 20,000 days of “baseline” diet by maximizing the Healthy Eating Patterns as described above with no other rules applied. This “baseline” diet is considered to be a healthy and balanced diet (Table 1). In this Example, the baselines were simulated for 1,400 individuals of each gender, ranging in age from 18 to 70, of varying heights and weights and sedentary physical activity level, for 7 days each. While the use of meals extracted from NHANES forces the simulated diets to use meals reportedly consumed by actual people, the optimization of the balance of food groups produces diets with a bias towards healthier diets than what people are likely to actually eat. As such we considered these to be “ideal” diets rather than realistic diets, which will tend to have higher nutrient intakes than occur in the real world.

TABLE 1
Percent of individuals with inadequate intakes
at baseline (from diet simulation models).

| | Males | | | Females | | |
|---|---|---|---|---|---|---|
| | Mean Intake | | Percent | Mean Intake | | Percent |
| Nutrient | Baseline | | Inadequate | Baseline | | Inadequate |
| Total fat | 47.1 | g | 0 | 41.7 | g | 0 |
| Fiber | 28.9 | g | 77 | 27.4 | g | 17 |
| Calcium | 1240.1 | mg | 9 | 1090.7 | mg | 41 |
| Copper | 1.7 | μg | 0 | 1.5 | μg | 0 |
| Food folate | 387.4 | μg | 12 | 376.0 | μg | 30 |
| Iron | 14.3 | mg | 0 | 12.2 | mg | 3 |
| Magnesium | 402.3 | mg | 2 | 370.8 | mg | 4 |
| Niacin | 20.9 | mg | 0 | 17.4 | mg | 7 |
| Potassium | 5121.9 | mg | 0 | 4620.0 | mg | 0 |
| Riboflavin | 2.3 | mg | 0 | 2.0 | mg | 0 |
| Selenium | 93.2 | μg | 0 | 77.4 | μg | 9 |
| Sodium | 2256.2 | mg | 1 | 1931.4 | mg | 24 |
| Vitamin A | 1076.1 | μg | 9 | 875.3 | μg | 23 |
| Vitamin B12 | 4.2 | μg | 2 | 3.3 | μg | 17 |
| Vitamin B6 | 3.0 | mg | 0 | 2.6 | mg | 1 |
| Vitamin C | 496.4 | mg | 0 | 479.5 | mg | 0 |
| Vitamin D | 7.5 | μg | 59 | 5.6 | μg | 86 |
| Vitamin E | 8.2 | mg | 94 | 8.0 | mg | 93 |
| Vitamin K | 113.7 | μg | 80 | 122.2 | μg | 52 |
| Zinc | 10.6 | mg | 18 | 9.0 | mg | 25 |

It is noted that some nutrients are low at baseline, meaning they are not at risk particularly because of IF diets, but in general in US diet/baseline.

### ADF Protocol Diet Simulation

The ADF protocols were created from the baseline diets by removing meals as per the rules of each ADF protocol:

Strict Alternate Day Fasting: every other day no food at all, with energy increased by 25% to mimic the increased energy intake typically consumed on eating days,

Modified Alternate Day Fasting: every other day all meals but lunch are removed, with lunch meal restricted to 400-600 kcal per day, and

5:2 on days 4 and 7 of every week all meals but breakfast are removed (to leave about 25% of daily calories, equivalent to about 500 kcal for someone consuming 2000 kcal/day).

By comparing the baseline inadequacies to the different ADF protocols, we can identify how nutrient intakes tend to change by following the various protocols (Table 2). This method of simulation, where a baseline diet is modified to comply with the rules of the IF protocol, forces the results to be comparable, allowing us to identify the relative risks of nutrient inadequacy for the various protocols while controlling for randomness inherent in the simulation process and for nutrient inadequacies which may result from the simulation process rather than being inherent in the ADF regimen.

TABLE 2
Percent of individuals with inadequate intakes when rules for
each ADF regimen were applied (from diet simulation models).

| | Percent of | | | Percent of | | |
|---|---|---|---|---|---|---|
| | Inadequacy, Males | | | Inadequacy, Females | | |
| | Strict | Modified | | Strict | Modified | |
| Nutrient | ADF | ADF | 5:2 | ADF | ADF | 5:2 |
| Total fat | 0.0 | 0.0 | 0.0 | 5.8 | 2.3 | 2.5 |
| Fiber | 98.9 | 97.0 | 97.5 | 85.4 | 76.6 | 71.0 |
| Calcium | 33.6 | 28.8 | 24.4 | 62.1 | 54.8 | 54.8 |
| Copper | 0.0 | 0.0 | 0.0 | 4.3 | 4.0 | 3.0 |
| Food Folate | 77.9 | 50.4 | 60.4 | 74.9 | 60.8 | 59.5 |
| Iron | 1.1 | 0.6 | 0.9 | 23.9 | 17.0 | 15.3 |
| Magnesium | 83.6 | 76.8 | 63.4 | 46.3 | 36.0 | 32.4 |
| Niacin | 8.8 | 3.6 | 5.0 | 34.4 | 21.3 | 21.1 |
| Potassium | 3.1 | 3.3 | 0.9 | 2.2 | 1.6 | 0.8 |
| Riboflavin | 2.9 | 3.3 | 2.0 | 8.9 | 7.4 | 6.8 |
| Selenium | 2.6 | 1.7 | 1.1 | 34.6 | 24.0 | 23.2 |
| Sodium | 9.0 | 5.4 | 6.1 | 59.9 | 45.9 | 45.1 |
| Vitamin A | 17.6 | 17.1 | 13.1 | 36.0 | 31.7 | 31.8 |
| Vitamin B12 | 10.0 | 7.6 | 6.6 | 43.4 | 37.6 | 40.4 |
| Vitamin B6 | 0.5 | 0.6 | 0.1 | 10.1 | 8.9 | 7.2 |
| Vitamin C | 0.0 | 0.0 | 0.0 | 0.0 | 0.0 | 0.0 |
| Vitamin D | 90.4 | 89.0 | 85.0 | 98.7 | 95.8 | 94.9 |
| Vitamin E | 98.9 | 99.1 | 98.1 | 98.5 | 98.0 | 97.9 |
| Vitamin K | 92.7 | 92.1 | 92.1 | 70.5 | 67.5 | 63.9 |
| Zinc | 67.4 | 61.9 | 57.0 | 5.9 | 45.5 | 45.8 |

The diet for each simulated individual is analyzed by comparing the mean intakes of each nutrient to the DRI for that individual, based on their age and gender. As nutrient intakes tend to not be normally distributed, bootstrap confidence intervals are created for the mean which are compared to the appropriate DRI. In order to pool results for individuals with varying DRIs, the result for each individual is calculated as a ratio of their mean intake to their DRI. The overall inadequacies/gaps between simulated intake and DRI are created by taking the mean of these ratios. The risks of inadequacies are created by taking the percentage of simulated individuals with inadequate intake for each nutrient.

The results of the analyses and the resulting recommendations for each group are below. These tables show the complete results of the analysis.

### Risk of Inadequate Intakes by ADF Regimen

Tables 3-8 show the risk for inadequacies identified for males and females for each ADF regimen. Most nutrients have an EAR, but when there were not sufficient data to assign an EAR, an AI was assigned instead (Institute of Medicine, US). In the Tables below, “Mean Inadequacy” is the amount below EAR or AI. The “Percent Inadequate” indicates the percent of individuals below the EAR or AI. If a large portion of the modelled diets produce inadequacies, then those nutrients are considered to be “At Risk” for inadequacies. Note that the interpretation is somewhat different for the AI; with a percent above an AI, we can generally assume adequacy, but a percent below does not necessarily imply inadequacy. A negative Mean Inadequacy indicates that the intakes tend to be adequate.

TABLE 3
Nutritional Analysis of Strict ADF for Males.

| | DRI | | EAR/AI | Mean | Percent | Assess- |
|---|---|---|---|---|---|---|
| Nutrient | Type | Units | Value | Inadequacy | Inadequate | ment |
| Total fat | EAR | g | 20 | −17.5 | 0.0 | |
| Fiber | AI | g | 34.8 | 13.1 | 98.9 | At Risk |
| Calcium | EAR | mg | 860 | −132 | 33.6 | At Risk |
| Copper | EAR | mcg | 0.7 | −0.6 | 0.0 | |
| Food folate | EAR | mcg | 320 | 35.8 | 77.9 | At Risk |
| Iron | EAR | mg | 6 | −5.7 | 1.1 | |
| Magnesium | EAR | mg | 346 | 36.1 | 83.6 | At Risk |
| Niacin | EAR | mg | 12 | −5.5 | 8.8 | |
| Potassium | AI | mg | 3000 | −1010 | 3.1 | |
| Riboflavin | EAR | mg | 1.1 | −0.78 | 2.9 | |
| Selenium | EAR | mcg | 45 | −32.8 | 2.6 | |
| Sodium | AI | mg | 1500 | −344 | 9.0 | |
| Vitamin A | EAR | mcg | 625 | −283 | 17.6 | |
| Vitamin B12 | EAR | mcg | 2. | −1.7 | 10.0 | |
| Vitamin B6 | EAR | mg | 1.22 | −1.25 | 0.5 | |
| Vitamin C | EAR | mg | 75 | −290.7 | 0.0 | |
| Vitamin D | EAR | mcg | 10 | 3.3 | 90.4 | At Risk |
| Vitamin E | EAR | mg | 12 | 6.0 | 98.9 | At Risk |
| Vitamin K | AI | mcg | 120 | 44.5 | 92.7 | At Risk |
| Zinc | EAR | mg | 9.4 | 0.7 | 67.4 | At Risk |

TABLE 4
Nutritional Analysis of Strict ADF for Females.

| | DRI | | EAR/AI | Mean | Percent | Assess- |
|---|---|---|---|---|---|---|
| Nutrient | Type | Units | Value | Inadequacy | Inadequate | ment |
| Total fat | EAR | g | 18.4 | −11.3 | 5.8 | |
| Fiber | AI | g | 23.4 | 3.8 | 85.4 | At Risk |
| Calcium | EAR | mg | 880 | 101 | 62.1 | At Risk |
| Copper | EAR | mcg | 0.7 | −0.4 | 4.3 | |
| Food folate | EAR | mcg | 320 | 50.8 | 74.9 | At Risk |
| Iron | EAR | mg | 6.9 | −1.8 | 23.9 | |
| Magnesium | EAR | mg | 263 | −2.2 | 46.3 | At Risk |
| Niacin | EAR | mg | 11 | −1.4 | 34.4 | At Risk |
| Potassium | AI | mg | 2300 | −1003 | 2.2 | At Risk |
| Riboflavin | EAR | mg | 0.9 | −0.50 | 8.9 | |
| Selenium | EAR | mcg | 45 | −10.3 | 34.6 | At Risk |
| Sodium | AI | mg | 1500 | 121 | 59.9 | |
| Vitamin A | EAR | mcg | 500 | −127 | 36.0 | At Risk |
| Vitamin B12 | EAR | mcg | 2 | −0.3 | 43.4 | At Risk |
| Vitamin B6 | EAR | mg | 1.18 | −0.67 | 10.1 | |
| Vitamin C | EAR | mg | 60 | −282.8 | 0.0 | |
| Vitamin D | EAR | mcg | 10 | 6.0 | 98.7 | At Risk |
| Vitamin E | EAR | mg | 12 | 6.3 | 98.5 | At Risk |
| Vitamin K | AI | mcg | 90 | 2.3 | 70.5 | At Risk |
| Zinc | EAR | mg | 6.8 | 0.4 | 5.9 | At Risk |

TABLE 5
Nutritional Analysis of Modified ADF for Males.

| | DRI | | EAR/AI | Mean | Percent | Assess- |
|---|---|---|---|---|---|---|
| Nutrient | Type | Units | Value | Inadequacy | Inadequate | ment |
| Total fat | EAR | g | 20 | −20.6 | 0.0 | |
| Fiber | AI | g | 34.8 | 12.5 | 97.0 | At Risk |
| Calcium | EAR | mg | 860 | −166 | 28.8 | At Risk |
| Copper | EAR | mcg | 0.7 | −0.6 | 0.0 | |
| Food folate | EAR | mcg | 320 | 16.2 | 50.4 | At Risk |
| Iron | EAR | mg | 6 | −5.9 | 0.6 | |
| Magnesium | EAR | mg | 346 | 25.0 | 76.8 | At Risk |
| Niacin | EAR | mg | 12 | −5.7 | 3.6 | |
| Potassium | AI | mg | 3000 | −1126 | 3.3 | |
| Riboflavin | EAR | mg | 1.1 | −0.8 | 3.3 | |
| Selenium | EAR | mcg | 45 | −40.3 | 1.7 | |
| Sodium | AI | mg | 1500 | −542 | 5.4 | |
| Vitamin A | EAR | mcg | 625 | −413 | 17.1 | |
| Vitamin B12 | EAR | mcg | 2. | −1.6 | 7.6 | |
| Vitamin B6 | EAR | mg | 1.22 | −1.3 | 0.6 | |
| Vitamin C | EAR | mg | 75 | −305.2 | 0.0 | |
| Vitamin D | EAR | mcg | 10 | 3.0 | 89.0 | At Risk |
| Vitamin E | EAR | mg | 12 | 5.5 | 99.1 | At Risk |
| Vitamin K | AI | mcg | 120 | 40.7 | 92.1 | At Risk |
| Zinc | EAR | mg | 9.4 | 0.4 | 61.9 | At Risk |

TABLE 6
Nutritional Analysis of Modified ADF for Females.

| | DRI | | EAR/AI | Mean | Percent | Assess- |
|---|---|---|---|---|---|---|
| Nutrient | Type | Units | Value | Inadequacy | Inadequate | ment |
| Total fat | EAR | g | 18.4 | −15.3 | 2.3 | |
| Fiber | AI | g | 23.4 | 2.7 | 76.6 | At Risk |
| Calcium | EAR | mg | 880 | 32 | 54.8 | At Risk |
| Copper | EAR | mcg | 0.7 | −0.5 | 4.0 | |
| Food folate | EAR | mcg | 320 | 26.0 | 60.8 | At Risk |
| Iron | EAR | mg | 6.9 | −2.4 | 17.0 | |
| Magnesium | EAR | mg | 263 | −22.9 | 36.0 | At Risk |
| Niacin | EAR | mg | 11 | −2.5 | 21.3 | At Risk |
| Potassium | AI | mg | 2300 | −1246 | 1.6 | At Risk |
| Riboflavin | EAR | mg | 0.9 | −0.6 | 7.4 | |
| Selenium | EAR | mcg | 45 | −17.6 | 24.0 | At Risk |
| Sodium | AI | mg | 1500 | −72.1 | 45.9 | |
| Vitamin A | EAR | mcg | 500 | −216 | 31.7 | At Risk |
| Vitamin B12 | EAR | mcg | 2 | −0.5 | 37.6 | At Risk |
| Vitamin B6 | EAR | mg | 1.18 | −0.8 | 8.9 | |
| Vitamin C | EAR | mg | 60 | −301.9 | 0.0 | |
| Vitamin D | EAR | mcg | 10 | 5.7 | 95.8 | At Risk |
| Vitamin E | EAR | mg | 12 | 5.7 | 98.0 | At Risk |
| Vitamin K | AI | mcg | 90 | −10.7 | 67.5 | At Risk |
| Zinc | EAR | mg | 6.8 | −0.3 | 45.5 | At Risk |

TABLE 7
Nutritional Analysis of the 5:2 ADF Regimen for Males.

| | DRI | | EAR/AI | Mean | Percent | Assess- |
|---|---|---|---|---|---|---|
| Nutrient | Type | Units | Value | Inadequacy | Inadequate | ment |
| Total fat | EAR | g | 20 | −19.7 | 0.0 | |
| Fiber | AI | g | 34.8 | 11.2 | 97.5 | At Risk |
| Calcium | EAR | mg | 860 | −227 | 24.4 | At Risk |
| Copper | EAR | mcg | 0.7 | −0.7 | 0.0 | |
| Food folate | EAR | mcg | 320 | 17.4 | 60.4 | At Risk |
| Iron | EAR | mg | 6 | −7.2 | 0.9 | |
| Magnesium | EAR | mg | 346 | 13.8 | 63.4 | At Risk |
| Niacin | EAR | mg | 12 | −7.0 | 5.0 | |
| Potassium | AI | mg | 3000 | −1290 | 0.9 | |
| Riboflavin | EAR | mg | 1.1 | −1.0 | 2.0 | |
| Selenium | EAR | mcg | 45 | −36.5 | 1.1 | |
| Sodium | AI | mg | 1500 | −432 | 6.1 | |
| Vitamin A | EAR | mcg | 625 | −344 | 13.1 | |
| Vitamin B12 | EAR | mcg | 2. | −2.3 | 6.6 | |
| Vitamin B6 | EAR | mg | 1.22 | −1.5 | 0.1 | |
| Vitamin C | EAR | mg | 75 | −312.6 | 0.0 | |
| Vitamin D | EAR | mcg | 10 | 2.6 | 85.0 | At Risk |
| Vitamin E | EAR | mg | 12 | 5.5 | 98.1 | At Risk |
| Vitamin K | AI | mcg | 120 | 41.5 | 92.1 | At Risk |
| Zinc | EAR | mg | 9.4 | −0.01 | 57.0 | At Risk |

TABLE 8
Nutritional Analysis of the 5:2 ADF Regimen for Females.

| | DRI | | EAR/AI | Mean | Percent | Assess- |
|---|---|---|---|---|---|---|
| Nutrient | Type | Units | Value | Inadequacy | Inadequate | ment |
| Total fat | EAR | g | 18.4 | −15.6 | 2.5 | |
| Fiber | AI | g | 23.4 | 2.1 | 71.0 | At Risk |
| Calcium | EAR | mg | 880 | 29.1 | 54.8 | At Risk |
| Copper | EAR | mcg | 0.7 | −0.5 | 3.0 | |
| Food folate | EAR | mcg | 320 | 20.3 | 59.5 | At Risk |
| Iron | EAR | mg | 6.9 | −2.5 | 15.3 | |
| Magnesium | EAR | mg | 263 | −29.4 | 32.4 | At Risk |
| Niacin | EAR | mg | 11 | −2.7 | 21.1 | At Risk |
| Potassium | AI | mg | 2300 | −1337 | 0.8 | At Risk |
| Riboflavin | EAR | mg | 0.9 | −0.6 | 6.8 | |
| Selenium | EAR | mcg | 45 | −18.1 | 23.2 | At Risk |
| Sodium | AI | mg | 1500 | −92.0 | 45.1 | |
| Vitamin A | EAR | mcg | 500 | −207.0 | 31.8 | At Risk |
| Vitamin B12 | EAR | mcg | 2 | −0.5 | 40.4 | At Risk |
| Vitamin B6 | EAR | mg | 1.18 | −0.8 | 7.2 | |
| Vitamin C | EAR | mg | 60 | −314.1 | 0.0 | |
| Vitamin D | EAR | mcg | 10 | 5.7 | 94.9 | At Risk |
| Vitamin E | EAR | mg | 12 | 5.6 | 97.9 | At Risk |
| Vitamin K | AI | mcg | 90 | −12.3 | 63.9 | At Risk |
| Zinc | EAR | mg | 6.8 | −0.3 | 45.8 | At Risk |

In this Example, the nutrients identified as “At Risk” were similar by ADF regimen but differed for Males and Females. However, the magnitude of the nutrient gaps differed by regimen.

### Analysis: Distributions of Intakes

Tables 9-14 show the distributions of intakes for the nutrients of interest. The nutrients of interest are selected as those which have a risk of inadequacy large enough to be notable, and where the percentage of simulated diets which have inadequate intakes differs significantly from the baseline.

TABLE 9
Distribution of Intakes for Nutrients of Risk for Strict ADF Regimen, Males.

| Nutrient | Units | EAR/AI | RDA | Minimum | 2.5% | 50% | 97.5% | Maximum |
|---|---|---|---|---|---|---|---|---|
| Calcium | mg | 860 | 1000 | 240.8 | 435.9 | 1074.2 | 3050.1 | 4070.8 |
| Fiber | g | 34.8 | NA | 8.1 | 14.1 | 29.7 | 52.3 | 78.1 |
| Food Folate | mcg | 320 | 400 | 60.6 | 137.3 | 362.1 | 715.8 | 852.6 |
| Magnesium | mg | 346 | 416 | 160.0 | 229.4 | 461.1 | 668.7 | 769.1 |
| Vitamin D | mcg | 10 | 15 | 0.1 | 0.9 | 5.7 | 22.6 | 88.6 |
| Vitamin E | mg | 12 | 15 | 2.6 | 3.8 | 7.6 | 18.7 | 41.6 |
| Vitamin K | mcg | 120 | NA | 12.0 | 22.4 | 58.6 | 344.6 | 2231.2 |
| Zinc | mg | 9.4 | 11 | 2.8 | 4.4 | 10.3 | 25.0 | 48.7 |

TABLE 10
Distribution of Intakes for Nutrients of Risk for Strict ADF Regimen, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 170.2 | 302.5 | 720.7 | 2761.8 | 4063.1 |
| Fiber | g | 23.4 | NA | 5.9 | 11.2 | 25.9 | 46.9 | 62.1 |
| Food Folate | mcg | 320 | 400 | 59.8 | 119.7 | 293.2 | 709.5 | 862.7 |
| Magnesium | mg | 263 | 318 | 138.5 | 166.3 | 355.0 | 612.3 | 739.1 |
| Niacin | mg | 11 | 14 | 3.7 | 5.7 | 14.6 | 34.3 | 90.2 |
| Potassium | mg | 2300 | NA | 1754.4 | 2168.2 | 4370.7 | 8140.3 | 9481.9 |
| Selenium | mcg | 45 | 55 | 14.5 | 22.9 | 57.9 | 151.6 | 335.1 |
| Vitamin A | mcg | 500 | 700 | 29.3 | 123.1 | 404.5 | 2004.4 | 3008.2 |
| Vitamin B12 | mcg | 2.0 | 2.4 | 0.1 | 0.3 | 1.6 | 8.6 | 23.6 |
| Vitamin D | mcg | 10 | 15 | 0.0 | 0.2 | 0.8 | 17.5 | 29.3 |
| Vitamin K | mcg | 90 | NA | 7.5 | 17.1 | 48.0 | 397.9 | 747.7 |
| Zinc | mg | 6.8 | 8.0 | 2.0 | 2.9 | 6.6 | 17.1 | 43.2 |

TABLE 11
Distribution of Intakes for Nutrients of Risk for Modified ADF Regimen, Males.

| Nutrient | Units | EAR/AI | RDA | Minimum | 2.5% | 50% | 97.5% | Maximum |
|---|---|---|---|---|---|---|---|---|
| Calcium | mg | 860 | 1000 | 82.7 | 126.3 | 859.3 | 2440.0 | 3256.6 |
| Fiber | g | 34.8 | NA | 2.6 | 6.9 | 23.8 | 41.9 | 62.5 |
| Food Folate | mcg | 320 | 400 | 16.1 | 16.1 | 289.7 | 572.6 | 682.1 |
| Magnesium | mg | 346 | 416 | 69.0 | 119.6 | 368.9 | 535.0 | 615.2 |
| Vitamin D | mcg | 10 | 15 | 0.0 | 0.1 | 5.5 | 18.7 | 70.9 |
| Vitamin E | mg | 12 | 15 | 1.0 | 2.4 | 6.3 | 15.0 | 33.3 |
| Vitamin K | mcg | 120 | NA | 4.5 | 17.6 | 53.9 | 279.3 | 1784.9 |
| Zinc | mg | 9.4 | 11 | 1.8 | 1.8 | 8.4 | 20.1 | 38.9 |

TABLE 12
Distribution of Intakes for Nutrients of Risk for Modified ADF Regimen, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 5.9 | 85.1 | 761.6 | 2180.2 | 3250.4 |
| Fiber | g | 23.4 | NA | 0.0 | 3.6 | 22.0 | 36.8 | 49.7 |
| Food Folate | mcg | 320 | 400.0 | 0.0 | 25.6 | 303.0 | 553.0 | 690.1 |
| Magnesium | mg | 263 | 318 | 22.1 | 50.5 | 307.3 | 480.7 | 591.3 |
| Niacin | mg | 11 | 14 | 0.3 | 1.2 | 13.9 | 26.8 | 72.2 |
| Potassium | mg | 2300 | NA | 245.4 | 735.9 | 3691.5 | 6348.0 | 7585.5 |
| Selenium | mcg | 45 | 55 | 0.3 | 1.1 | 61.6 | 120.7 | 268.1 |
| Vitamin A | mcg | 500 | 700 | 0.0 | 12.0 | 680.9 | 1582.9 | 2444.5 |
| Vitamin B12 | mcg | 2.0 | 2.4 | 0.0 | 0.0 | 2.2 | 6.8 | 18.9 |
| Vitamin D | mcg | 10 | 15 | 0.0 | 0.0 | 3.5 | 14.5 | 23.5 |
| Vitamin K | mcg | 90 | NA | 0.0 | 1.6 | 67.1 | 326.8 | 598.1 |
| Zinc | mg | 6.8 | 8.0 | 0.2 | 0.4 | 7.2 | 13.4 | 34.5 |

TABLE 13
Distribution of Intakes for Nutrients of Risk for 5:2, Males.

| Nutrient | Units | EAR/AI | RDA | Minimum | 2.5% | 50% | 97.5% | Maximum |
|---|---|---|---|---|---|---|---|---|
| Calcium | mg | 860 | 1000 | 30.6 | 30.6 | 1095.1 | 2489.4 | 3256.6 |
| Fiber | g | 34.8 | NA | 1.1 | 3.3 | 26.8 | 43.0 | 59.8 |
| Food Folate | mcg | 320 | 400 | 4.5 | 4.5 | 355.3 | 589.8 | 682.1 |
| Magnesium | mg | 346 | 416 | 12.9 | 12.9 | 402.5 | 541.0 | 629.0 |
| Vitamin D | mcg | 10 | 15 | 0.0 | 0.0 | 6.7 | 19.3 | 70.9 |
| Vitamin E | mg | 12 | 15 | 0.1 | 0.3 | 6.9 | 16.3 | 36.7 |
| Vitamin K | mcg | 120 | NA | 0.6 | 1.4 | 65.7 | 295.5 | 1784.9 |
| Zinc | mg | 9.4 | 11 | 0.1 | 0.1 | 10.0 | 22.0 | 40.7 |

TABLE 14
Distribution of Intakes for Nutrients of Risk for 5:2, Females.

| Nutrient | Units | EAR/AI | RDA | Minimum | 2.5% | 50% | 97.5% | Maximum |
|---|---|---|---|---|---|---|---|---|
| Calcium | mg | 880 | 1080 | 5.9 | 83.7 | 796.7 | 2136.6 | 3250.5 |
| Fiber | g | 23.4 | NA | 0.0 | 3.6 | 22.2 | 36.4 | 49.7 |
| Food Folate | mcg | 320 | 400 | 0.0 | 23.6 | 318.1 | 546.6 | 690.1 |
| Magnesium | mg | 263 | 318 | 22.1 | 49.9 | 312.7 | 475.2 | 591.3 |
| Niacin | mg | 11 | 14 | 0.3 | 1.1 | 14.2 | 26.2 | 72.2 |
| Potassium | mg | 2300 | NA | 245.4 | 745.1 | 3775.8 | 6250.9 | 7585.6 |
| Selenium | mcg | 45 | 55 | 0.3 | 1.0 | 60.7 | 120.4 | 268.1 |
| Vitamin A | mcg | 500 | 700 | 0.0 | 9.9 | 677.2 | 1569.5 | 2444.5 |
| Vitamin B12 | mcg | 2.0 | 2.4 | 0.0 | 0.0 | 2.2 | 6.8 | 18.9 |
| Vitamin D | mcg | 10 | 15 | 0.0 | 0.0 | 3.3 | 14.7 | 23.5 |
| Vitamin K | mcg | 90 | NA | 0.0 | 1.0 | 72.7 | 329.6 | 598.1 |
| Zinc | mg | 6.8 | 8.0 | 0.2 | 0.3 | 7.3 | 13.3 | 34.5 |

### Analysis: Amount of Inadequacy

Tables 15-20 show the amount below the EAR or AI for each percentile (2.5%, 50% and 97.5%) for the nutrients of interest. The nutrients of interest are selected as those which have a risk of inadequacy large enough to be notable, and where the percentage of simulated diets which have inadequate intakes differs significantly from the baseline. If the values are negative it means that the intake tends to be above the DRI.

TABLE 15
Amount of inadequacy of each “At Risk”
nutrient for Strict ADF for Males.

| Nutrient | Units | EAR/AI | 2.5% Gap | 50% Gap | 97.5% Gap |
|---|---|---|---|---|---|
| Calcium | mg | 860 | 424.1 | −214.2 | −2190.1 |
| Fiber | g | 34.8 | 20.7 | 5.1 | −17.5 |
| Food Folate | mcg | 320 | 182.7 | −42.1 | −395.8 |
| Magnesium | mg | 346 | 116.6 | −115.2 | −322.7 |
| Vitamin D | mcg | 10.0 | 9.1 | 4.3 | −12.6 |
| Vitamin E | mg | 12.0 | 8.2 | 4.4 | −6.7 |
| Vitamin K | mcg | 120 | 97.6 | 61.4 | −224.6 |
| Zinc | mg | 9.4 | 5.0 | −0.9 | −15.6 |

TABLE 16
Amount of inadequacy of each “At Risk”
nutrient for Strict ADF for Females.

| Nutrient | Units | EAR/AI | 2.5% Gap | 50% Gap | 97.5% Gap |
|---|---|---|---|---|---|
| Calcium | mg | 880 | 577.5 | 159.3 | −1881.8 |
| Fiber | g | 23.4 | 12.2 | −2.5 | −23.5 |
| Food Folate | mcg | 320 | 200.3 | 26.8 | −389.5 |
| Magnesium | mg | 263 | 96.7 | −92.0 | −349.3 |
| Niacin | mg | 11.0 | 5.3 | −3.6 | −23.3 |
| Potassium | mg | 2300 | 131.8 | −2070.8 | −5840.4 |
| Selenium | mcg | 45 | 22.1 | −12.9 | −106.6 |
| Vitamin A | mcg | 500 | 376.9 | 95.6 | −1504.4 |
| Vitamin | mcg | 2.0 | 1.6 | 0.4 | −6.6 |
| B12 | | | | | |
| Vitamin D | mcg | 10.0 | 9.8 | 9.2 | −7.5 |
| Vitamin K | mcg | 90 | 72.9 | 42.0 | −307.9 |
| Zinc | mg | 6.8 | 3.9 | 0.2 | −10.3 |

TABLE 17
Amount of inadequacy of each “At Risk”
nutrient for Modified ADF for Males.

| Nutrient | Units | EAR/AI | 2.5% Gap | 50% Gap | 97.5% Gap |
|---|---|---|---|---|---|
| Calcium | mg | 860 | 733.7 | 0.6 | −1580.1 |
| Fiber | g | 34.8 | 27.9 | 11.0 | −7.1 |
| Food Folate | mcg | 320 | 303.9 | 30.3 | −252.6 |
| Magnesium | mg | 346 | 226.3 | −22.9 | −189.0 |
| Vitamin D | mcg | 10.0 | 9.9 | 4.5 | −8.7 |
| Vitamin E | mg | 12.0 | 9.6 | 5.7 | −3.0 |
| Vitamin K | mcg | 120 | 102.4 | 66.1 | −159.3 |
| Zinc | mg | 9.4 | 7.6 | 1.0 | −10.7 |

TABLE 18
Amount of inadequacy of each “At Risk”
nutrient for Modified ADF for Females.

| Nutrient | Units | EAR/AI | 2.5% Gap | 50% Gap | 97.5% Gap |
|---|---|---|---|---|---|
| Calcium | mg | 880 | 794.9 | 118.4 | −1300.2 |
| Fiber | g | 23.4 | 19.8 | 1.4 | −13.4 |
| Food Folate | mcg | 320 | 294.3 | 17.0 | −233.0 |
| Magnesium | mg | 263 | 212.5 | −44.3 | −217.7 |
| Niacin | mg | 11.0 | 9.8 | −2.9 | −15.8 |
| Potassium | mg | 2300 | 1564.0 | −1391.6 | −4048.1 |
| Selenium | mcg | 45 | 43.9 | −16.6 | −75.7 |
| Vitamin A | mcg | 500 | 488.0 | −180.8 | −1082.9 |
| Vitamin | mcg | 2.0 | 2.0 | −0.2 | −4.8 |
| B12 | | | | | |
| Vitamin D | mcg | 10.0 | 10.0 | 6.5 | −4.5 |
| Vitamin K | mcg | 90 | 88.4 | 22.9 | −236.8 |
| Zinc | mg | 6.8 | 6.4 | −0.4 | −6.6 |

TABLE 19
Amount of inadequacy of each “At Risk”
nutrient for 5:2 for Males.

| Nutrient | Units | EAR/AI | 2.5% Gap | 50% Gap | 97.5% Gap |
|---|---|---|---|---|---|
| Calcium | mg | 860 | 829.4 | −235.1 | −1629.5 |
| Fiber | g | 34.8 | 31.5 | 8.0 | −8.2 |
| Food Folate | mcg | 320 | 315.5 | −35.3 | −269.8 |
| Magnesium | mg | 346 | 333.1 | −56.5 | −195. |
| Vitamin D | mcg | 10.0 | 10.0 | 3.3 | −9.3 |
| Vitamin E | mg | 12.0 | 11.7 | 5.1 | −4.3 |
| Vitamin K | mcg | 120 | 118.6 | 54.3 | −175.5 |
| Zinc | mg | 9.4 | 9.3 | −0.6 | −12.6 |

TABLE 20
Amount of inadequacy of each “At Risk”
nutrient for 5:2 for Females.

| Nutrient | Units | EAR/AI | 2.5% Gap | 50% Gap | 97.5% Gap |
|---|---|---|---|---|---|
| Calcium | mg | 880 | 796.3 | 83.3 | −1256.6 |
| Fiber | g | 23.4 | 19.8 | 1.2 | −13.0 |
| Food Folate | mcg | 320 | 296.4 | 1.9 | −226.6 |
| Magnesium | mg | 263 | 213.1 | −49.7 | −212.2 |
| Niacin | mg | 11.0 | 9.9 | −3.2 | −15.2 |
| Potassium | mg | 2300 | 1554.9 | −1475.8 | −3950.9 |
| Selenium | mcg | 45 | 44.0 | −15.7 | −75.4 |
| Vitamin A | mcg | 500 | 490.1 | −177.2 | −1069.5 |
| Vitamin | mcg | 2.0 | 2.0 | −0.2 | −4.8 |
| B12 | | | | | |
| Vitamin D | mcg | 10.0 | 10.0 | 6.7 | −4.7 |
| Vitamin K | mcg | 90 | 89.0 | 17.3 | −239.6 |
| Zinc | mg | 6.8 | 6.5 | −0.5 | −6.5 |

### Recommendations

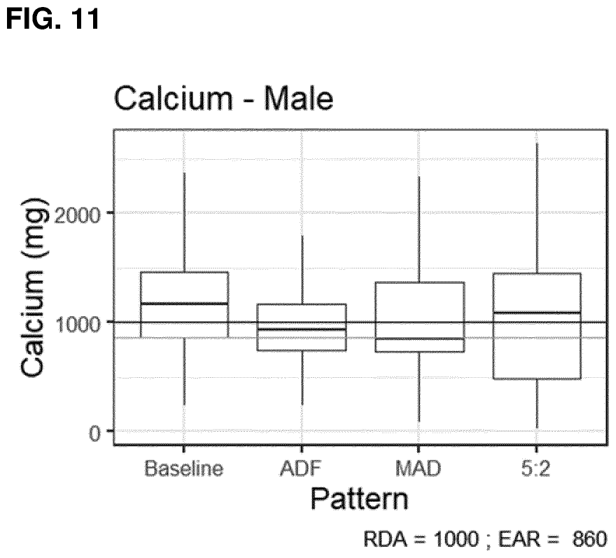

In order to construct dietary recommendations, we consider the upper and lower bounds of the intake recommendations to identify nutrient targets, without exceeding the UL (see FIGS. 11-14 for examples for calcium and fiber). The “Amount of Inadequacy” is used to generate the dietary recommendations. First, we take the difference between the RDA, or the AI if no RDA is available, and the 2.5% intake percentile as the upper and the difference with the 97.5% intake percentile as the lower. All recommendations are based on an energy level for the diet of 2000 kcal. The upper end of the recommendation is capped to ensure that the recommendation does not exceed the UL.

We then employ a computerized Diet Simulator. By making certain assumptions, the Diet Simulator can provide a reasonable estimate of the nutritional inadequacies/gaps and thus provide recommendations on how to close those gaps from nutrients, foods, beverages, and/or dietary supplements, and their combinations, without requiring undue effort or input from the user. In this Example, assumptions included the following:
The individual eats a typical meal pattern (e.g., breakfast, lunch, dinner and one snack per day),
The individual eats typical food and beverage combinations for those meals and snacks (e.g., meals and snacks as reported in NHANES), and
The individual intends to follow a well-balanced diet in accordance with dietary recommendations (e.g., the USDA Healthy Eating Pattern).

The food composition database used by the Diet Simulator contains the nutrient content, including macro- and micro-nutrients for each food per 100 g and per representative serving sizes. In this example, the USDA Food Data Central databases are used to identify foods and beverages containing sufficient levels of the nutrients identified as “At Risk” in the preceding steps. The Diet Simulator is also linked to a recipe database so that recipes which contain food sources of the nutrients “At Risk” can be identified

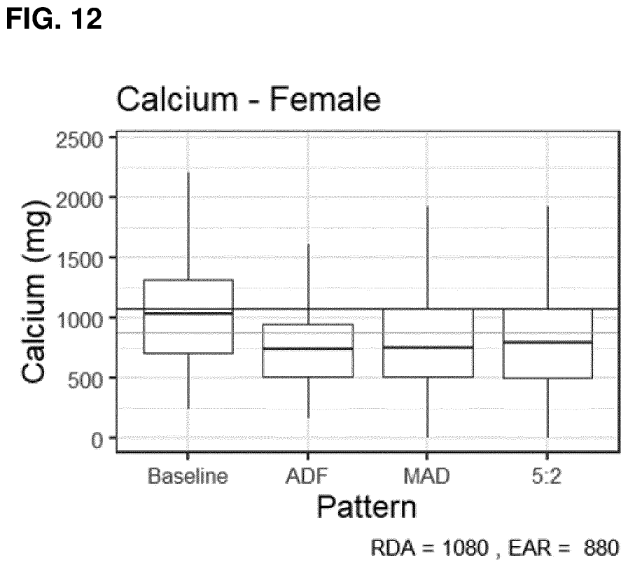

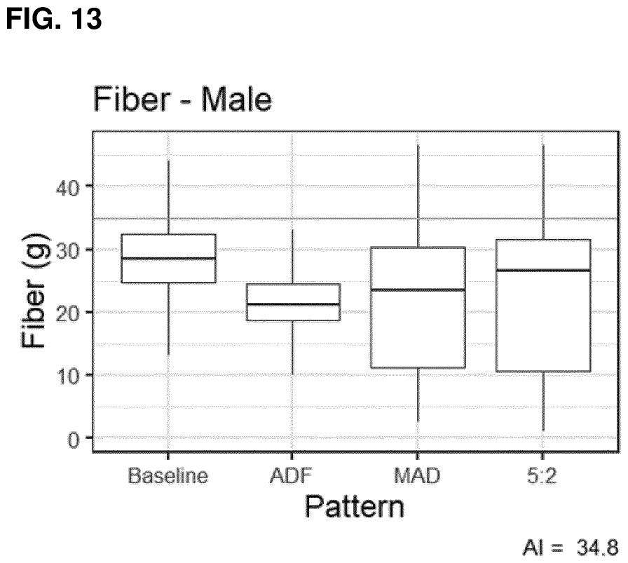

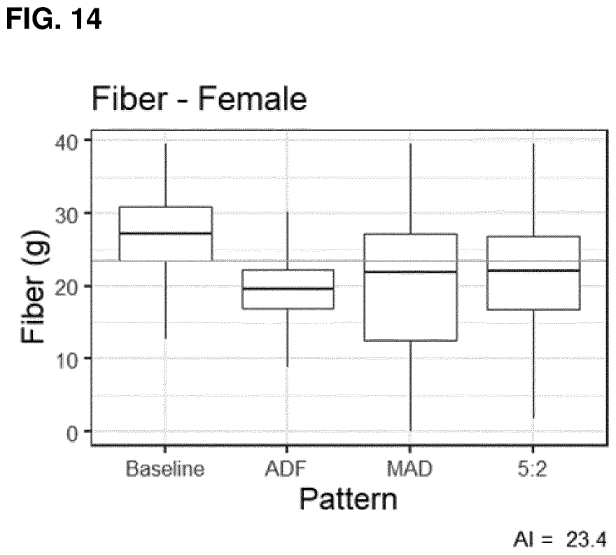

In this example, we have selected only two of these nutrients to exemplify recommendations. Thus, for e.g., fiber and calcium that were identified as nutrients of need for all of the ADF regimens. FIG. 11 shows calcium intake ranges comparing simulated baseline intakes with Strict ADF, Modified ADF and 5:2 for males. FIG. 12 shows calcium intake ranges comparing simulated baseline intake with Strict ADF, Modified ADF and 5:2 for females. FIG. 13 shows fiber intake ranges comparing simulated baseline intakes with Strict ADF, Modified ADF and 5:2 for males. FIG. 14 shows fiber intake ranges comparing simulated baseline intakes with Strict ADF, Modified ADF and 5:2 for females.

Examples, such as the following dietary recommendations would be generated to accompany feedback on each of the ADF diets. In a similar manner, dietary recommendations for all of the nutrients identified as “At Risk” could be generated. As an illustration, we provide examples for calcium and dietary fiber as follows:

### Calcium:

Calcium could be recommended from food sources. For example, someone specifying a vegetarian diet may receive a recommendation for soybean curd (tofu) cubes; 1 cup (248 g) provides 275 mg of calcium. Similarly, a recipe for tofu with mixed vegetables, including broccoli and carrots with a soy-based sauce could be recommended; 2 cups (434 g) provides 286 mg of calcium.

Calcium could be recommended from beverages. For example, someone specifying a flexitarian diet may receive a recommendation for low-fat milk; 1 cup (246 g) of low-fat milk contains 310 mg of calcium.

Calcium could be recommended as a dietary supplement. Non-limiting examples of suitable forms of calcium include one or more calcium salts, such as calcium acetate, calcium carbonate, calcium chloride, calcium citrate, calcium gluconate, calcium lactate or mixtures thereof.

The amount of additional calcium recommended would depend on the needs identified in the simulations. For example, the Strict ADF for men showed a deficit of 424 mg of calcium per day (Table 15). This amount could be provided by 336 g of milk, or about 1.4 cups.

### Fiber

Fiber could be recommended from food sources. For example, someone specifying a Vegan diet may receive a recommendation for cooked oatmeal; 1 cup (234 g) provides 4 mg of dietary fiber. Similarly, a recipe for oatmeal raisin cookies could be recommended; 2 cookies (48 g) provides 1.6 g of fiber.

Fiber could be recommended from dietary supplements. Non-limiting suitable forms of fiber supplements include those containing wheat dextrin, methylcellulose, psyllium husk, polycarbophil, guar fiber, glucomannan or β-glucans.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. (canceled)

2. The method according to claim 11, wherein the recommendations comprise diet recommendations, menu recommendations and recipe recommendations.

3-4. (canceled)

5. The method according to claim 11, wherein the intermittent fasting (IF) diet is a time-restricted feeding (TRF) regimen or an alternate day fasting (ADF) regimen.

6. The method according to claim 11, wherein the nutritional recommendations are determined by a process comprising generating a rule for each of a plurality of nutrients or ingredients, the rule generated by defining one or more of a desired lower bound of the rule, a desired upper bound of the rule, an absolute minimum for the rule, an absolute maximum for the rule, a weight for lower bound of the rule, or a weight for upper bound of the rule; the process further comprising generating a goal for each of the plurality of nutrients or ingredients, the goal is maximizing the amount of the corresponding nutrient or ingredient or minimizing the amount of the corresponding nutrient or ingredient.

7. The method according to claim 11, wherein the nutritional recommendations are determined using a database of meal items and pre-created meals, wherein the pre-created meals each comprise one or more of the meal items which form an entire meal, and each of the pre-created meals are associated with summary nutrition information for the entire meal; and the process of determining the nutritional recommendations comprises generating a variable for each of the pre-created meals which indicates whether the corresponding pre-created meal is included in a menu plan, based on comparison of the summary nutrition information to the rules and goals.

8. The method according to claim 11, wherein the nutritional recommendations are determined by generating two constraints for each rule, each of the two constraints comprising an upper bound and a lower bound, wherein each constraint is the sum of the amount of each food included in the menu plan, multiplied by the amount of the nutrient in the corresponding food, plus a corresponding slack variable which represents the deviation of the menu plan from the desired range for the corresponding nutrient.

9. The method according to claim 11, wherein the process of determining the nutritional recommendations comprises generating an objective term for the goals by summing over each goal and each food, the amount of the corresponding food included in the menu plan plus the amount of the corresponding nutrient in the food multiplied by the goal coefficient, normalized by the maximum amount of the corresponding nutrient in the foods in the database.

10. The method according to claim 11, wherein the process of determining the nutritional recommendations comprises creating a menu plan by minimizing the sum of the slack variables over the rules weighted by the corresponding weight of the rule, plus the objective term.

11. A method for providing nutritional recommendations to an individual before, during and/or after and intermittent fasting (IF) diet comprising using a computer-implemented system comprising:
a menu database module;
a recipe database module;
a dietary constraints module;
a nutrient scoring module; and
an optimisation module,
wherein the computer-implemented system is configured to generate nutritionally-healthy recommendations for the individual before, during and/or after an intermittent fasting (IF) diet.

12. The computer-implemented system according to claim 11, wherein the nutritionally-healthy recommendations are selected from the group comprising food recommendations, menu recommendations and recipe recommendations for a time-restricted feeding (TRF) regimen or an alternate day fasting (ADF) regimen.

13. The computer-implemented system according to claim 11, wherein the nutritionally-healthy recommendations comprise individualized nutritional recommendations based on at least one individual user attribute selected from the group consisting of age, gender, height, weight, BMI, medical condition, physical activity level, and total recommended daily energy allowance.

14. The method according to claim 11, wherein the nutritional recommendations comprise individualized nutritionally-healthy recommendations based on at least one dietary preference or constraint selected from the group consisting of omnivorous diet, gluten-free, lactose-free, Mediterranean, paleo, vegan, and vegetarian.

15. The method according to claim 11, wherein the nutritional recommendations comprise nutritionally-healthy recommendations for menus classified as suitable for a breakfast, a lunch, a dinner or a snack.

16. The method according to claim 11, wherein the nutritional recommendations comprise recipe recommendations.

17-18. (canceled)

19. A method of minimizing or preventing nutrient inadequacy in an individual performing an IF regimen, the method comprising:
using the computer-implemented system comprising:
a menu database module;
a recipe database module;
a dietary constraints module;
a nutrient scoring module; and
an optimisation module,
wherein the computer-implemented system is configured to generate nutritionally-healthy recommendations for the individual before, during and/or after an intermittent fasting (IF) diet to obtain the nutritionally-healthy recommendations; and
administering to the individual one or more of a nutrient, a food containing a nutrient, or an amount thereof, according to the nutritionally-healthy recommendations from the computer-implemented system, the administration being performed to the individual before, during and/or after the IF diet.

20. The method of claim 19 wherein at least one of a food comprising fiber, a supplement selected from the group consisting of fiber, an amount of a food or supplement comprising fiber, a food comprising calcium, a supplement comprising calcium, or an amount of a food and supplement comprising calcium is administered to the individual.
